(19) United States
(12) Patent Application Publication
(10) Pub. No.: US 2025/0311950 A1
(43) Pub. Date: Oct. 9, 2025

(54) MULTIFUNCTIONAL HYDROGEL MICRONEEDLE ELECTRODE FOR KETONE SENSING

(71) Applicants: Mahla Poudineh, Waterloo (CA); Amin GhavamI Nejad, Waterloo (CA); Irfani Rahmi Ausri, Waterloo (CA); Sadegh Sadeghzadeh, Waterloo (CA)

(72) Inventors: Mahla Poudineh, Waterloo (CA); Amin GhavamI Nejad, Waterloo (CA); Irfani Rahmi Ausri, Waterloo (CA); Sadegh Sadeghzadeh, Waterloo (CA)

(21) Appl. No.: 19/170,784

(22) Filed: Apr. 4, 2025

Related U.S. Application Data
(60) Provisional application No. 63/731,173, filed on Apr. 8, 2024.

(51) Int. Cl. A61B 5/1473; A61B 5/00; A61B 5/145

(52) U.S. Cl. CPC A61B 5/14735; A61B 5/14514; A61B 5/14532; A61B 5/14546; A61B 5/6833; A61B 5/685; A61B 5/7475; A61B 2562/0215; A61B 2562/046; A61B 2562/125

(57) ABSTRACT

The present disclosure relates to a microneedle analyte sensing device for continuous monitoring of an analyte in a user's biological fluid, specifically designed for detecting ketone levels in diabetic ketoacidosis (DKA) management. The disclosed microneedle analyte sensing device utilizes dopamine (DA) molecules covalently linked to the microneedle patch's polymer structure or toluidine blue O (TBO), serving as a redox mediator for measuring the oxidation byproduct of 3-beta-hydroxybutyrate (β-HB). The sensing mechanism relies on catechol-quinone chemistry, where a pre-oxidation approach correlates sensor response to β-HB concentrations. The device comprises a plurality of microneedles on a substrate that, when applied to the skin, penetrate the stratum corneum to contact biological fluid. At least one microneedle functions as a working electrode to detect an electrochemical signal from the enzymatic reaction with the analyte, while additional microneedles serve as counter and reference electrodes to facilitate accurate electrochemical measurements.

### CROSS-REFERENCE TO RELATED APPLICATIONS

This application claims benefit of and priority to U.S. Provisional Application No. 63/731,173, filed on Apr. 8, 2024, the entire contents of which are incorporated by reference herein for all purposes.

### COLOR DRAWINGS

The patent or application file contains at least one drawing executed in color. Copies of this patent or patent application publication with color drawing(s) will be provided by the Office upon request and payment of the necessary fee.

### BACKGROUND

### 1. Field of the Invention

The disclosure provides biosensors or biological analyte sensing devices for continuously monitoring and measuring a biomarker or an analyte in a biological fluid of a user.

### 2. Brief Description of the Related Art

Diabetic ketoacidosis (DKA), a life-threatening complication of type 1 diabetes (TID), is characterized by uncontrolled hyperglycemia and increased ketone body concentration. The two main ketone bodies are acetoacetate (AcAc) and 3-beta-hydroxybutyrate (β-HB). According to existing diabetes guidelines, a ketone level below 0.6 mM is categorized as within the normal range while levels between 0.6-1.5 mM are classified as ketosis. Levels between 1.6-3 mM signify a risk of DKA and any ketone levels above 3 mM are categorized as high and indicative of DKA. Despite major developments in continuous glucose monitor (CGM) devices, the need for continuous ketone bodies monitoring (CKM) devices remains limited. Current approaches for measuring ketone bodies rely on self-monitoring using commercially available blood or urine strips; these tools are poorly adopted and report only a single time point measurement.

Microneedles (MNs) are three-dimensional microstructures that can physically penetrate the stratum corneum, enabling transdermal sensing of biomarkers within interstitial fluid (ISF) in a minimally invasive manner. Traditional MN biosensors utilize solid and rigid MNs as the device electrodes, including the recently reported-HB sensors. Abbott Inc. has also recently reported a solid needle-based CKM device that uses the same technology as their CGM device. However, the solid MNs/needles are incompatible with the mechanically soft and curved skin, leading to progressive retraction of the MNs and diminished signal over time. To prevent signal retraction, previously, subcutaneous placement of magnetic plates via skin incision was employed, making the deployment of solid MNs invasive and unsuitable for human usage. Furthermore, solid MNs made from materials such as metals, are biologically incompatible, and their application may trigger immune responses or tissue reactions when inserted into the skin. Needle breakage within the skin is also another critical concern. If breakage occurs, recovery of the broken needles can be a challenging and invasive process. Rectifying the significant mismatch in physical properties, using functional polymers and flexible biomaterials holds the potential to advance biocompatible systems that seamlessly integrate with human skin.

Compared to the commonly used oxidase enzymes, the β-hydroxybutyrate dehydrogenase (HBD) enzyme requires nicotinamide adenine dinucleotide (NAD⁺) as a cofactor to catalyze the oxidation of β-HB to produce AcAc and 3-nicotinamide adenine dinucleotide (NADH). However, detection of β-HB through direct NADH oxidation requires a high potential (˜1V), which can cause surface fouling and non-specific detection of interfering analytes. Therefore, redox mediators are integrated to lower the detection potential and facilitate a fast transfer rate. Quinones derivatives, such as phenanthroline quinones, and 5,5-dihydroxy-4,4-bitryptamine, have been reported to perform effectively as redox mediators for NADH oxidation. Specifically, 1,10-phenanthroline-5,6-dione (PD) was used in dehydrogenase-based sensors for detecting glucose, lactate, and ketone bodies. Another quinones derivative, toluidine blue O (TBO), has also been reported as a redox mediator for β-HB detection. The stable immobilization of these redox mediators along with the NAD⁺ co-factor and enzyme must be achieved towards the development of robust dehydrogenase-based sensors.

Dopamine (DA) possesses redox-active catechol and quinone moieties that have previously been explored for NADH sensing. However, it has been underutilized in enzymatic biosensing potentially due to the reduced presence of the redox-active quinone moieties at physiological pH (pKa=8.9).

### SUMMARY

Diabetic ketoacidosis, a severe complication of type 1 diabetes (T1D) is triggered by production of large quantities of ketone bodies, requiring patients with TID to constantly monitor their ketone levels. The present disclosure describes a microneedle analyte sensing device for continuously monitoring and measuring an analyte in a biological fluid of a user.

In one aspect, disclosed herein is a microneedle analyte sensing device, which comprises a plurality of microneedles operable to penetrate a surface of a biological tissue of the user and contact the plurality of microneedles with the biological fluid when the microneedle analyte sensing device is attached to the surface of the biological tissue (e.g., skin or stratum corneum). At least one microneedle of the plurality microneedles is a working electrode that detects an electrical signal generated from an electrochemically mediated enzymatic reaction with the analyte in the biological fluid of the user, at least one microneedle of the plurality of microneedles is a counter electrode, and at least one microneedle of the plurality of microneedles is a reference electrode. In some embodiments, the working electrode, the counter electrode and the reference electrode are incorporated into a single microneedle array to generate a wearable continuous ketone monitoring device.

In any aspect or embodiment described herein, at least one of: the at least one microneedle that is the working electrode is made of a hydrogel; the at least one microneedle that is the counter electrode is made of ultraviolet (UV)-cured epoxy and coated with metal nanoparticles, wherein the metal nanoparticles comprise platinum, silver, gold, palladium, or combinations thereof; the at least one microneedle that is the reference electrode is made of ultraviolet (UV)-cured epoxy and coated with silver-silver chloride (Ag/AgCl); or a combination thereof.

In any aspect or embodiment described herein, at least one of: the hydrogel comprises at least one of hyaluronic acid, methacrylated hyaluronic acid, gelatin, methacrylated gelatin, alginate, methacrylated alginate, chitosan, methacrylated chitosan, collagen, methacrylated collagen, or a combination thereof; the at least one microneedle that is the working electrode comprises enzymes integrated in the hydrogel, and wherein the enzymes comprise at least one of beta-hydroxybutyrate dehydrogenase, tyrosinase, or a combination thereof; the at least one microneedle that is the working electrode comprises a HBD cofactor integrated in the hydrogel, wherein the HBD cofactor comprises nicotinamide adenine dinucleotide (NAD+); the at least one microneedle that is the working electrode comprises a redox mediator integrated in the hydrogel to facilitate electron transfer in the electrochemically mediated enzymatic reaction; the at least one microneedle that is the working electrode comprises an electrically conductive material integrated in the hydrogel to increase electrical conductivity of the at least one microneedle; or a combination thereof.

In any aspect or embodiment described herein, the microneedle analyte sensing device is detects the electrical signal generated from the electrochemically mediated enzymatic reaction in situ.

In any aspect or embodiment described herein, the analyte is ketone, acetoacetate, beta-hydroxybutyrate, lactate, acetone, or glucose.

In any aspect or embodiment described herein, the biological fluid is interstitial fluid, transdermal fluid, extracellular fluid, or blood.

In any aspect or embodiment described herein, at least one of: the plurality of microneedles are disposed on a substrate or within a substrate that the plurality of microneedles are operable to penetrate; the microneedle analyte sensing device is integrated into a transdermal patch; the biological tissue is skin or stratum corneum; or a combination thereof.

In any aspect or embodiment described herein, at least one of: the redox mediator comprises at least one of dopamine, conjugated dopamine, functionalized dopamine, crosslinked dopamine, metal-complexed dopamine, poly toluidine blue O (PTBO), toluidine blue O (TBO), or combinations thereof; the electrically conductive material comprises at least one of poly(3,4-ethylenedioxythiophene): polystyrene sulfonate (PEDOT:PSS), a metal nanoparticle, graphene, MXene, conductive polymer, polyaniline, polypyrrole, ionomer, carbon nano tube, or a combination thereof; the electrochemically mediated enzymatic reaction is detectable using amperometry, impedimetry, conductometry, voltammetry, or potentiometry; or a combination thereof.

In any aspect or embodiment described herein, the microneedle analyte sensing device is a continuous ketone monitoring (CKM) sensor.

In another aspect, disclosed herein is an analyte sensing device comprising: the microneedle analyte sensing device of the present disclosure, and an electrical circuit electrically comprising a data processing unit, wherein: the electrical circuit is connected to the microneedle analyte sensing device and processes (e.g., programmed to process) the electrical signal generated by the electrochemically mediated enzymatic reaction; and the data processing unit comprises a processor and a memory, and processes (e.g., configured to process) the electrical signal as data representative of one or more parameters of the analyte.

In any aspect or embodiment described herein, the analyte sensing device further comprises a wireless communication unit in communication with the electrical circuit to transmit a processed signal to a user interface, wherein the user interface comprises a smartphone, a personal computer, a laptop, a tablet, a wearable device, a smart home device, an Internet of Things (IoT) device, or a combination thereof.

In any aspect or embodiment described herein, the analyte sensing device is a continuous ketone monitoring (CKM) sensor.

In another aspect, disclosed herein is a method for measuring an analyte within a biological fluid of a user. The method includes providing a microneedle analyte sensing device, wherein the microneedle analyte sensing device comprises a plurality of microneedles operable to penetrate a surface of a biological tissue of the user and contact the plurality of microneedles with the biological fluid when the microneedle analyte sensing device is attached to the surface of the biological tissue (e.g., skin or stratum corneum); placing the microneedle analyte sensing device on the surface of the biological tissue of the user to contact (e.g., transdermally contact) the plurality of microneedles with the biological fluid; applying an electrical stimulus signal to at least one microneedle of the plurality of microneedles; detecting an electrical signal arising by an electrochemically mediated enzymatic reaction with the analyte in the biological fluid of the user exposed to the at least one microneedle; and determining a concentration of the analyte based on the electrical signal.

In any aspect or embodiment described herein, at least one microneedle of the plurality of microneedles is a working electrode that detects the electrical signal generated from the electrochemically mediated enzymatic reaction with the analyte in the biological fluid of the user; at least one microneedle of the plurality of microneedles is a counter electrode; and at least one microneedle of the plurality of microneedles is a reference electrode.

In any aspect or embodiment described herein, at least one of: the plurality of microneedles are disposed on a substrate or within a substrate that the plurality of microneedles are operable to penetrate; the microneedle analyte sensing device is integrated into a transdermal patch; the biological tissue is skin or stratum corneum; the electrical signal is transferred through the at least one microneedle to an electrical circuit; or a combination thereof.

In any aspect or embodiment described herein, the method further includes at least one of: sending the electrical signal from the electrical circuit to a data processing unit, wherein the data processing unit comprises a processor and a memory and processes (e.g., configured to process) the electrical signal as data representative of one or more parameters of the analytes; sending the electrical signal from the data processing unit to a wireless communication unit in communication with the electrical circuit to transmit a processed signal to a user interface, wherein the user interface comprises a smartphone, a personal computer, a laptop, a tablet, a wearable device, a smart home device, a Internet of Things (IoT) device, or a combination thereof; or a combination thereof.

In an additional aspect, disclosed herein is a method of manufacturing a microneedle analyte sensing device. The method includes applying or injecting a composition for a working-electrode to a micro-mold for microneedles of a working electrode, wherein the composition for the working-electrode comprises a hydrogel, at least one enzyme, at least one enzyme cofactor, a redox mediator, and an electrically conductive material.

In any aspect or embodiment described herein, the method further comprises preparing the composition for the working-electrode by mixing the hydrogel, the at least one enzyme, the at least one enzyme cofactor, the redox mediator, and the electrically conductive material.

In any aspect or embodiment described herein, at least one of: (i) the method further comprises preparing a counter electrode, comprising: applying or injecting ultraviolet (UV)-curable epoxy to a micro-mold for microneedles of the counter electrode; curing the UV-curable epoxy of the counter electrode, optionally via ultraviolet light; coating the counter electrode with metal nanoparticles, wherein the metal nanoparticles comprise platinum, silver, gold, palladium, or combinations thereof; (ii) the method further comprises preparing a reference electrode, comprising: applying or injecting ultraviolet (UV)-curable epoxy to a micro-mold for microneedles of the reference electrode; curing the UV-curable epoxy of the reference electrode, optionally via ultraviolet; and coating the reference electrode with silver-silver chloride (Ag/AgCl); (iii) the hydrogel comprises at least one of hyaluronic acid, methacrylated hyaluronic acid, gelatin, methacrylated gelatin, alginate, methacrylated alginate, chitosan, methacrylated chitosan, collagen, methacrylated collagen, or combinations thereof; (iv) the at least one enzyme comprise at least one of beta-hydroxybutyrate dehydrogenase (HBD), tyrosinase, or a combination thereof; (v) the at least one enzyme cofactor comprises nicotinamide adenine dinucleotide (NAD+); (vi) the redox mediator comprises dopamine, conjugated dopamine, functionalized dopamine, crosslinked dopamine, metal-complexed dopamine, poly toluidin blue O (PTBO), toluidine blue O (TBO), or combinations thereof; (vii) the electrically conductive material comprises at least one of poly(3,4-ethylenedioxythiophene): polystyrene sulfonate (PEDOT:PSS), a metal nanoparticle, graphene, MXene, conductive polymer, polyaniline, polypyrrole, ionomer, carbon nano tube, or a combination thereof; or (viii) a combination thereof.

In any aspect or embodiment described herein, at least one of: the microneedles of the working electrode are homogeneous throughout the microneedles; the microneedles of the counter electrode are homogeneous throughout the microneedles; the microneedles of the reference electrode are homogeneous throughout the microneedles; the method further comprises forming one or more molds corresponding to shapes of (i) the microneedles of the working electrode, (ii) the microneedles of the counter electrode, (iii) the microneedles of the reference electrode, or (iv) a combination thereof; or a combination thereof.

In an additional aspect, disclosed herein is a method of manufacturing a microneedle analyte sensing device. The manufacturing method comprises applying a first composition to an electrode, wherein the first composition comprises a redox mediator, chitosan, and an electrically conductive material; curing the first composition to generate a first layer; applying a second composition to the first layer to generate a second layer on top of the first layer, wherein the second composition comprises at least one enzyme and at least one enzyme cofactor; and applying a third composition to the second layer to generate a third layer on top of the second layer, wherein the third composition comprises chitosan.

In any aspect or embodiment described herein, at least one of: the method further comprises preparing the first composition by mixing the redox mediator, the chitosan, and the electrically conductive material; the method further comprises preparing the second composition by mixing the at least one enzyme and the at least one enzyme cofactor; the electrode is a screen printed gold electrode; the electrically conductive material comprises or is carbon nanotubes (e.g., multi-walled carbon nanotubes, single-walled carbon nanotubes, double-walled carbon nanotubes, or a combination thereof); curing the first composition via cyclic voltammetry to generate a first layer; the at least one enzyme comprise at least one of beta-hydroxybutyrate dehydrogenase

(HBD), tyrosinase, or a combination thereof; the at least one enzyme cofactor comprises nicotinamide adenine dinucleotide (NAD+);the redox mediator comprises at least one of dopamine, conjugated dopamine, functionalized dopamine, crosslinked dopamine, metal-complexed dopamine, poly toluidin blue O (PTBO), toluidine blue O (TBO), or combinations thereof; or a combination thereof.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure. Further objects, features and advantages of the disclosure will become apparent from the following detailed description taken in conjunction with the accompanying figures showing illustrative embodiments of the disclosure.

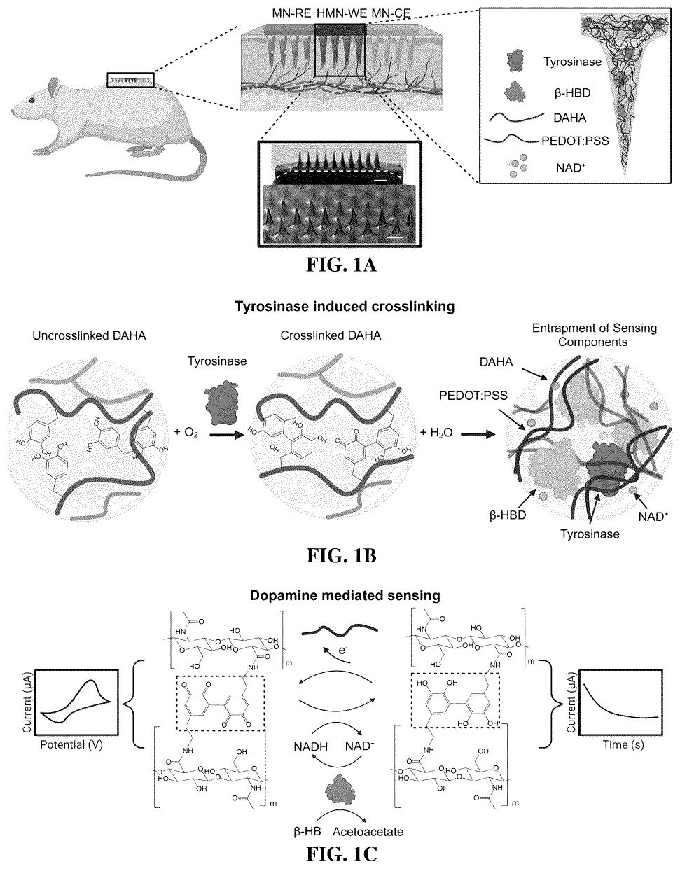

FIGS. 1A-C provide an overview of microneedle analyte sensing device. FIG. 1A: Schematic of the microneedle analyte sensing device applied on the back of a rat with a zoomed-in view of the hydrogel microneedle (HMN) working electrode (HMN-WE; scale bar=400 μm), Ag/AgCl microneedle reference electrode (MN-RE), and gold microneedle counter electrodes (MN-CE) and the magnified view of the HMN-WE composition. FIG. 1B: Illustration of the tyrosinase induced crosslinking of the DA-HA polymer in situ. FIG. 1C: Sensing mechanism of the microneedle analyte sensing device that leverages the DA's quinone and catechol chemistry to measure ketone bodies. All the schematics were created using BioRender, and chemical structures were drawn by ChemDraw.

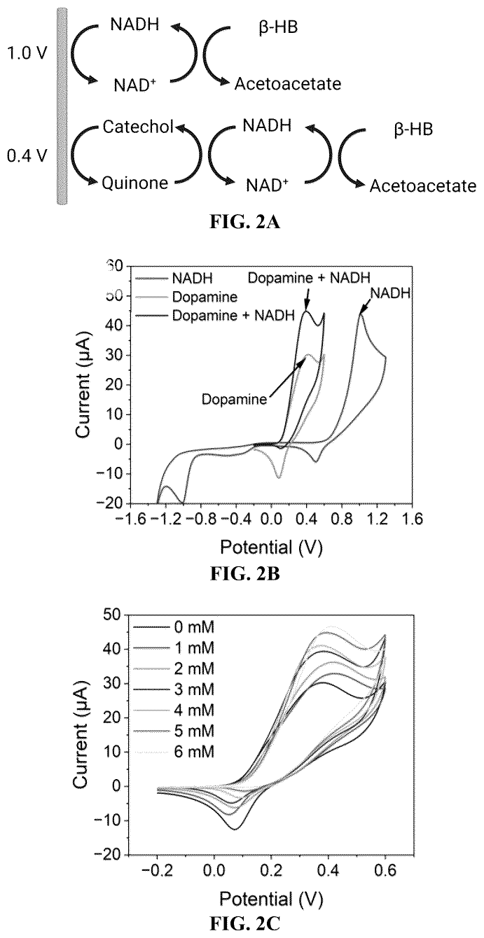

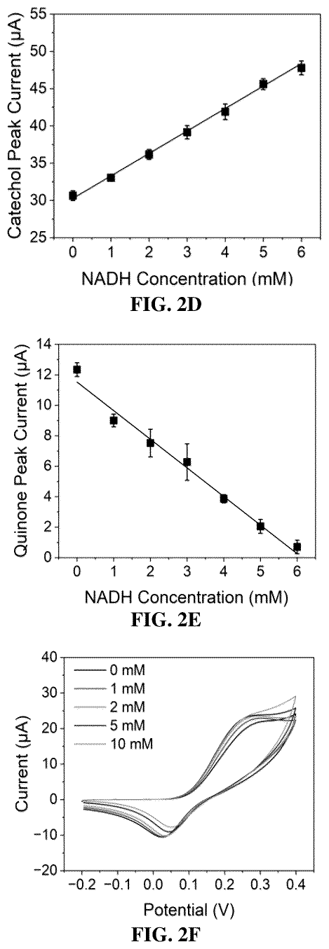

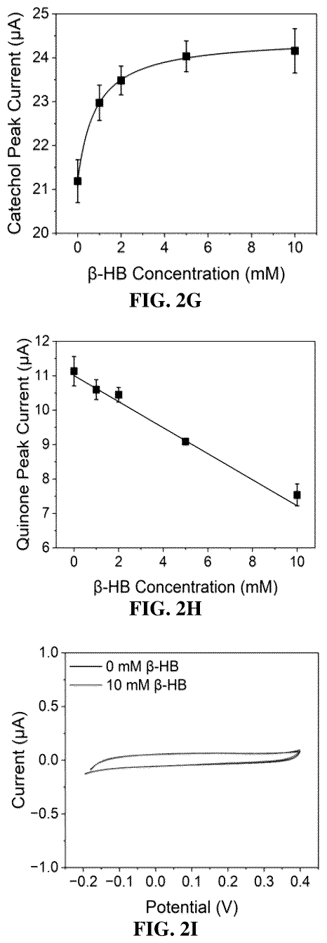

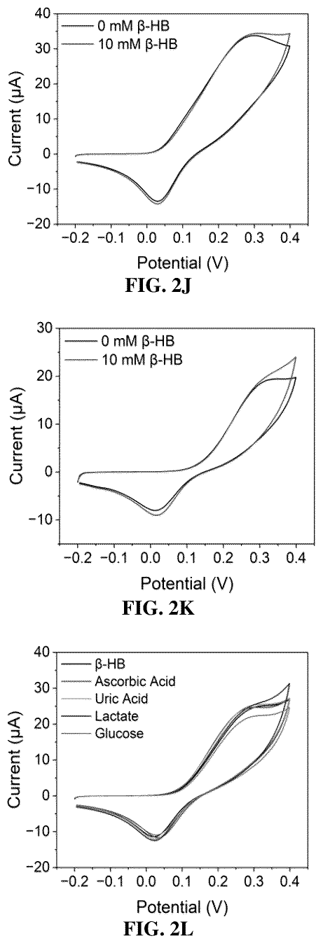

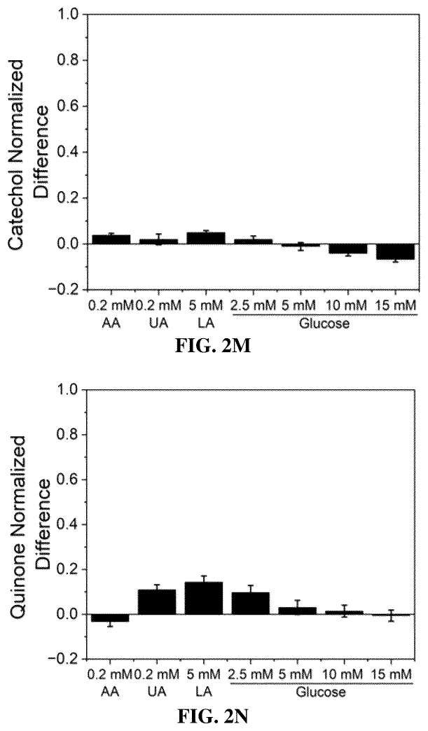

FIGS. 2A-2N show the validation of dopamine mediated sensing. FIG. 2A: Schematic of NADH sensing with and without DA mediation. FIG. 2B: Confirmation of role of DA as redox mediator in NADH sensing. In the presence of 5 mM NADH, the catechol oxidation peak increases while the quinone reduction peak decreases, indicating the electron transfer from NADH to quinone to produce NAD⁺ and catechol. FIG. 2C: NADH-DA interaction, Cyclic voltammetry (CV) curves of DA at different concentrations of NADH. FIG. 2D: Calibration curve of the catechol oxidation peaks at different concentrations of NADH. Error bars represent the standard deviation of n=3. FIG. 2E: Calibration curve of the quinone reduction peaks at different concentrations of NADH. Error bars represent the standard deviation of n=3. FIG. 2F: β-HB sensing, CV curves of DA in the presence of HBD, NAD⁺, and different concentrations of β-HB. G, Calibration curve of the catechol oxidation peaks at different concentrations of β-HB. Error bars represent the standard deviation of n=3. FIG. 2H: Calibration curve of the quinone reduction peaks at different concentrations of β-HB. Error bars represent the standard deviation of n=3. FIGS. 2I-K, CV curves of the experimental set up in the absence of DA (FIG. 2I), NAD⁺ (FIG. 2J), and HBD (FIG. 2K) in response to 0 and 10 mM β-HB. FIG. 2L: Specificity study, CV curves of DA in response to common physiological interferents. Normalized difference of FIG. 2M, catechol oxidation peak and FIG. 2N, quinone reduction peak in the presence of ascorbic acid (AA), uric acid (UA), lactate (LA), and glucose compared to 0 mM β-HB (n=3). Significance is determined through Dunnett's one-way ANOVA and shown in GP style (0.1234 (ns), 0.0332 (*), 0.0021 (**), 0.0002 (***), p<0.0001 (****)). CV scans were conducted with a scan rate of 0.05 V s⁻¹. The potential range for NADH and β-HB CV scans were −0.2-0.6 and −0.2-0.4 V, respectively. Experiments were performed using CTI electrodes.

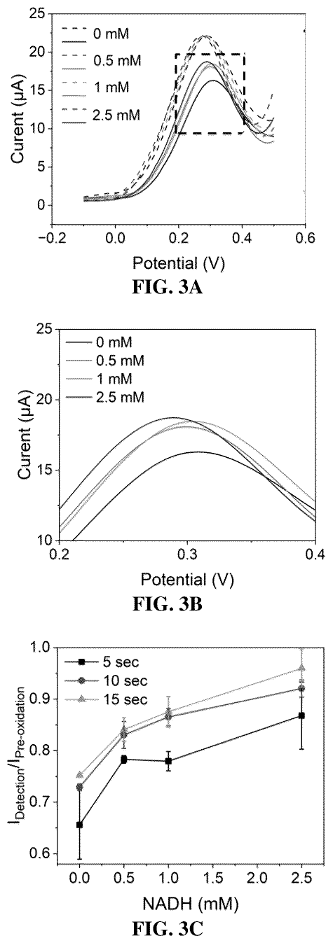

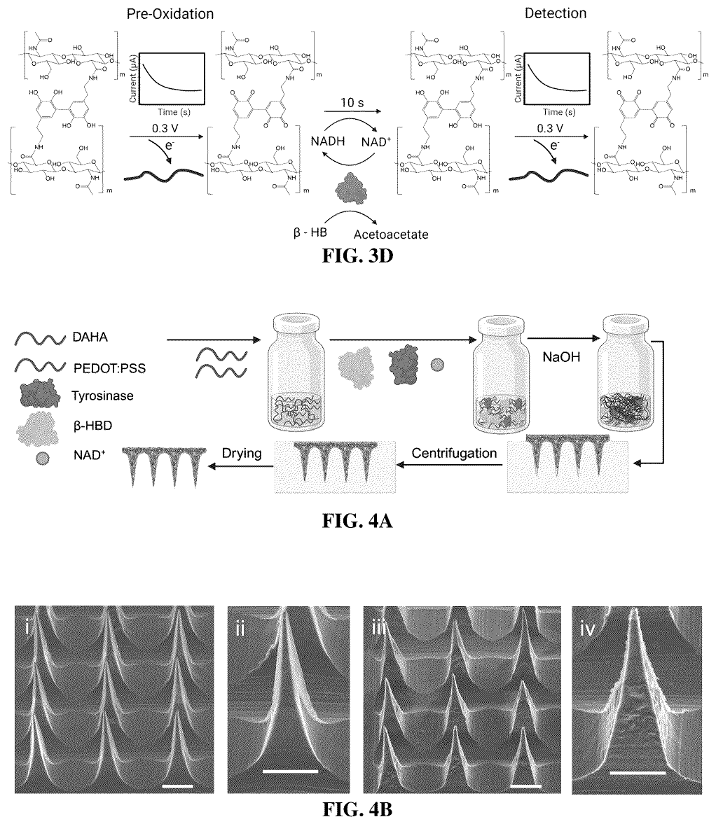

FIGS. 3A-3D show the validation of a universal approach for ketone sensing. FIG. 3A: Pre-oxidation (dashed) and detection (solid) SWV curves of DA at different NADH concentrations at 5-second wait time. Frequency=15 Hz. FIG. 3B: Zoomed-in SWV curves, from FIG. 3A, of the detection step of DA at different NADH concentrations. FIG. 3C: Effect of varying wait times on the catechol recovery (IDetection/IPre-oxidation) at different NADH concentrations. Error bars represent the standard deviation of n=3. FIG. 3D: Schematic of β-HB sensing protocol. The catechol redox signal detected in step 1 corresponds to all detectable catechol while the signal from step 3 represents the amount of catechol recovered after the quinone reacts with NADH. Therefore, the I_{Detection}/I_{Pre-oxidation} ratio represents the recovered catechol, which positively correlates with β-HB concentration. All the chemical structures were drawn by ChemDraw.

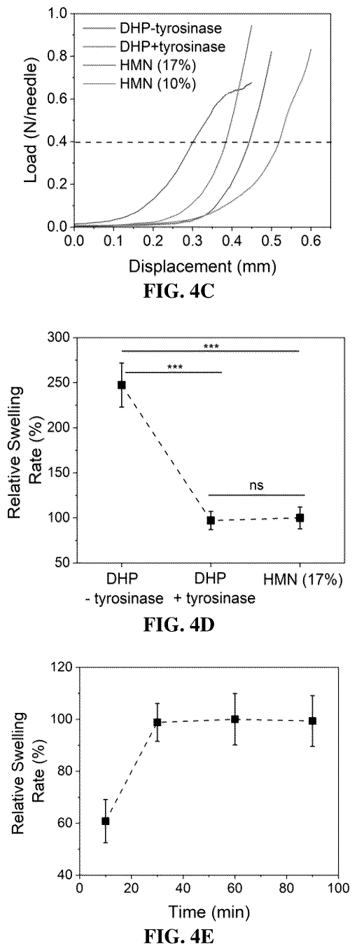

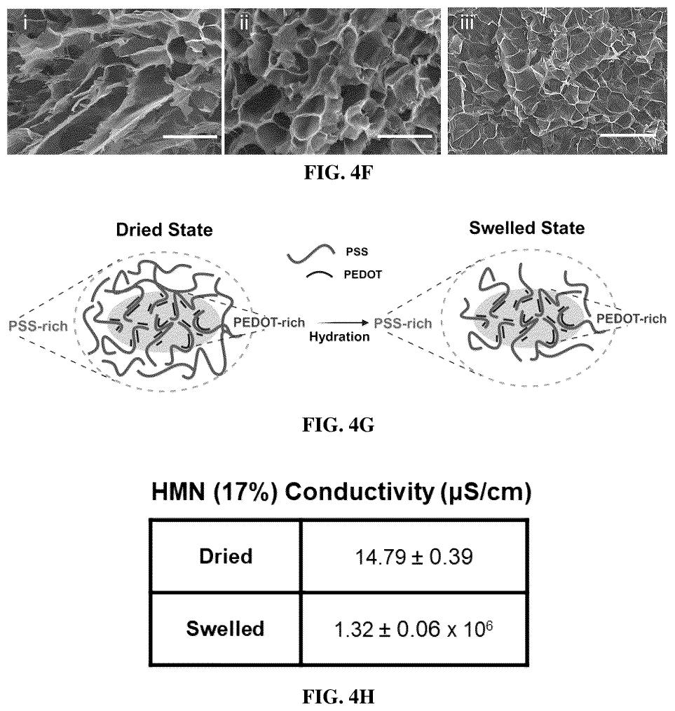

FIGS. 4A-4H show the microneedle analyte sensing device fabrication and characterization. FIG. 4A: Schematic of the fabrication process of microneedle analyte sensing device patches. FIG. 4B: Scanning electron microscopy (SEM) image of microneedle analyte sensing device patches: (i) HMN (17%), (ii) Zoomed-in image of one needle of HMN (17%), (iii) HMN (10%), and (iv) Zoomed-in image of one needle of HMN (10%) (scale bar=200 μm). FIG. 4C: Mechanical strength testing of DHP-tyrosinase, DHP+tyrosinase, HMN (17%), and HMN (10%). FIG. 4D: Relative swelling rate of DHP-tyrosinase, DHP+tyrosinase, and HMN (17%) corresponding to the swelling rate of HMN (17%). Error bars represent the standard deviation of n=3. Significance was shown in GP style (0.1234 (ns), 0.0332 (*), 0.0021 (**), 0.0002 (***), p<0.0001 (****)). FIG. 4E: Relative swelling rate of HMN (17%) at different time points corresponding to the maximum swelling rate of HMN (17%). Error bars represent the standard deviation of n=3. FIG. 4F: SEM images showing the porosity of different hydrogel composites: (i) DHP-tyrosinase, (ii) DHP+tyrosinase, and (iii) HMN 17% (scale bar=50 μm). FIG. 4G: Schematic illustrating removing extra PSS out of the hydrogel upon swelling. H, Electrical conductivity of HMN (17%) patches in dried and swelled states. All the schematics were created using BioRender.

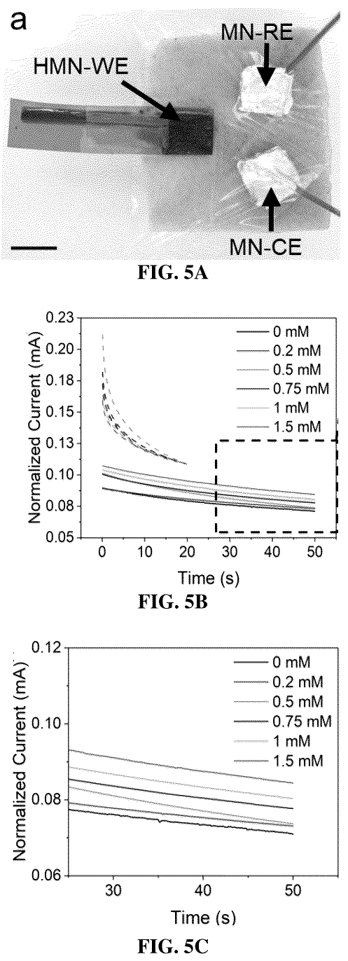

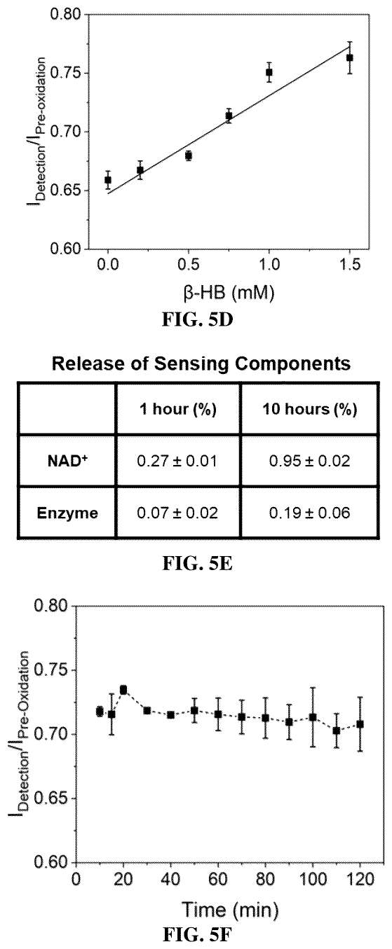

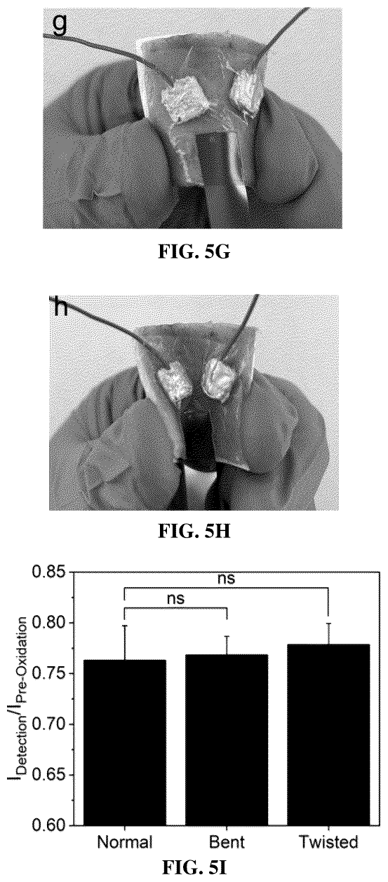

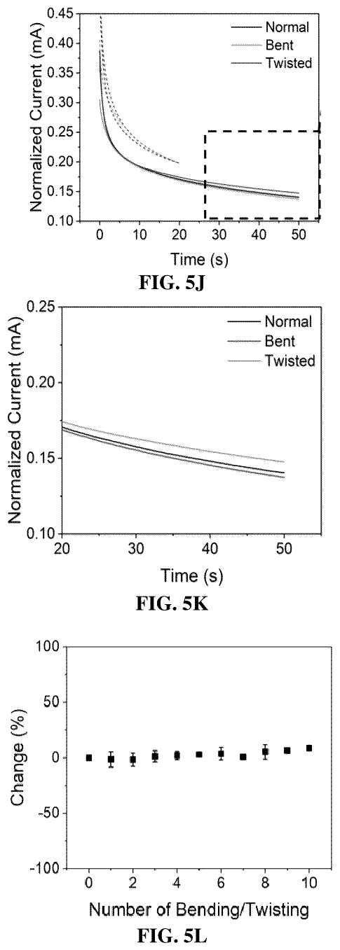

FIGS. 5A-5L show the validation of ketone sensing using an ex vivo skin model. FIG. 5A: Photograph of the ex vivo experimental setup with the porcine skin soaked at different concentrations of β-HB (scale bar=0.5 cm). FIG. 5B: Pre-oxidation (dashed) and detection (solid) chronoamperometry curves at 0.3V applied potential at different concentrations of β-HB. FIG. 5C: Magnified detection chronoamperometry curves, from FIG. 5B, at 0.3V applied potential at increasing concentration of β-HB. FIG. 5D: Calibration curve of microneedle analyte sensing device at different concentrations of β-HB. Error bars represent the standard deviation of (n=2−4). FIG. 5E: Table showing the NAD+ and HBD enzyme release after 1 or 10 hours of patch application on agarose hydrogel and its corresponding standard deviation (n=3). FIG. 5F: Stability study of the microneedle analyte sensing device in porcine skin soaked with 0.75 mM β-HB. Error bars represent the standard deviation of n=3. Mechanical stability test: FIGS. 5G-5H, Photograph of the microneedle analyte sensing device under twisted and bent states in porcine skin soaked with 1.5 mM β-HB. FIG. 5I: Sensor response at normal, bent, and twisted states. Error bars represent the standard deviation of n=3. Significance is determined through two-way ANOVA and shown in GP style (0.1234 (ns), 0.0332 (*), 0.0021 (**), 0.0002 (***), p <0.0001 (****)). FIG. 5J: Pre-oxidation (dashed) and detection (solid) chronoamperometry curves at 0.3V applied potential when the microneedle analyte sensing device was at normal, bent, and twisted states. FIG. 5K: Magnified detection chronoamperometry curves, from FIG. 5J, at 0.3V applied potential at normal, bent, and twisted states. FIG. 5L: Percent change in sensor response after successive bending and twisting cycles.

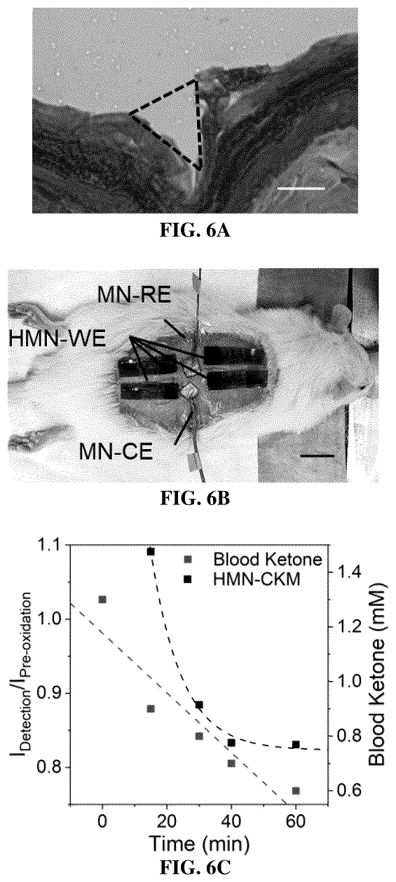

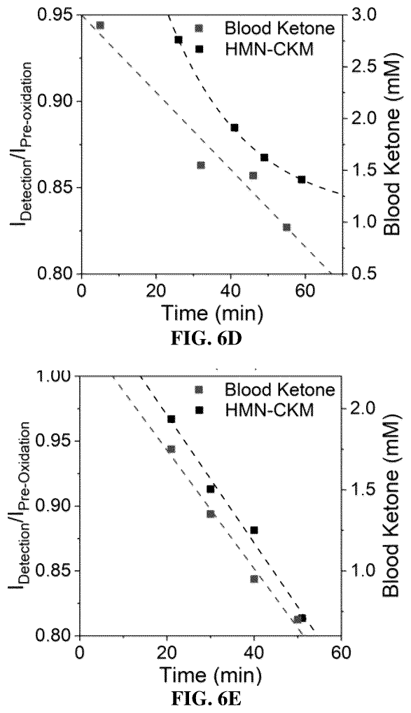

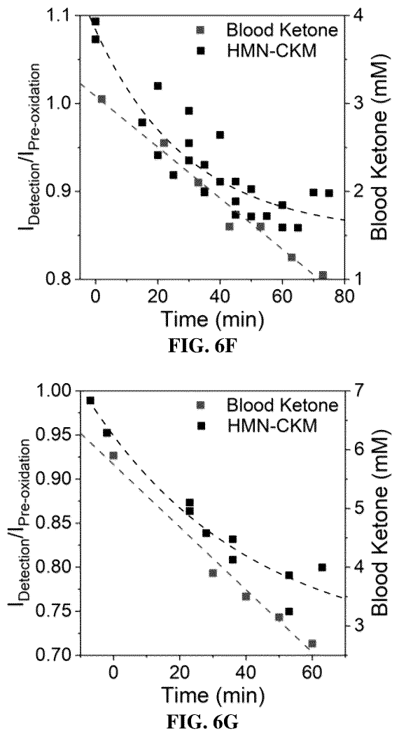

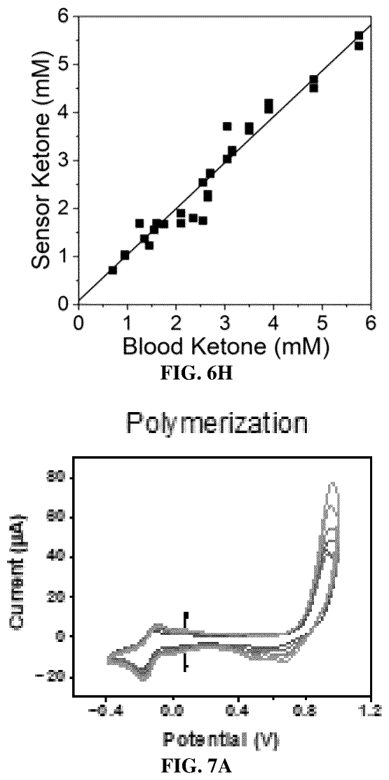

FIGS. 6A-6H show In vivo ketone sensing on diabetic rats. FIG. 6A: hematoxylin and eosin (H&E) staining on rat skin illustrating the needle cavity (scale bar=50 μm). FIG. 6B:

Photograph of the in vivo experimental setup with multiple HMN-WEs, MN-RE, and MN-CE placed on the dorsal back of a rat (scale bar=1 cm). FIG. 6C-G: microneedle analyte sensing device signal output and blood ketone obtained from commercial ketone strips showing decreasing ketone level upon insulin (10 U) injection in 5 rats. FIG. 6H: Correlation curve of the sensor ketone level predicted by the machine learning model against the reference blood ketone considering the time-lag (R²=0.95).

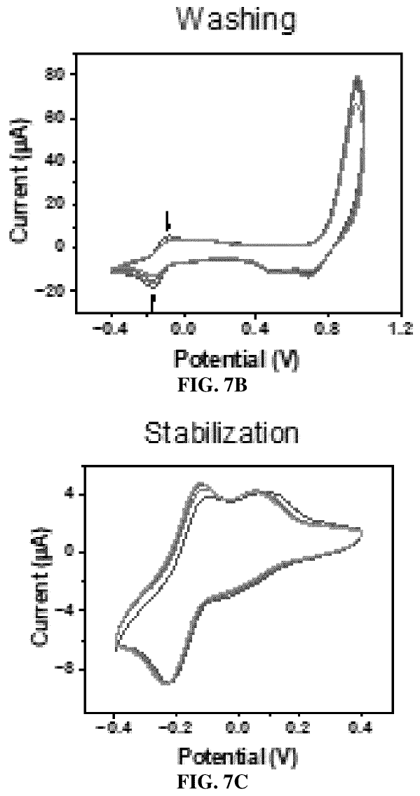

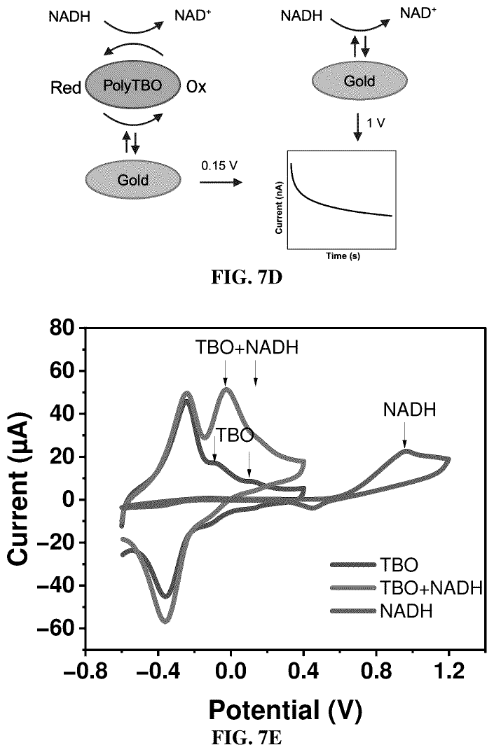

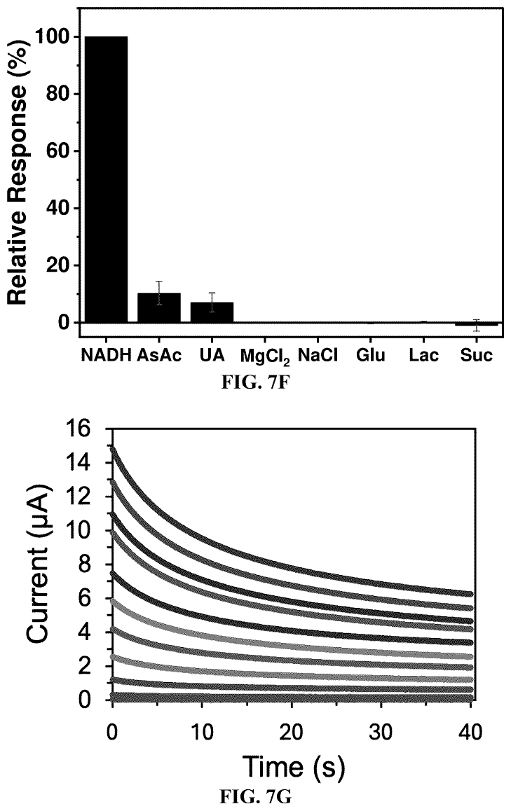

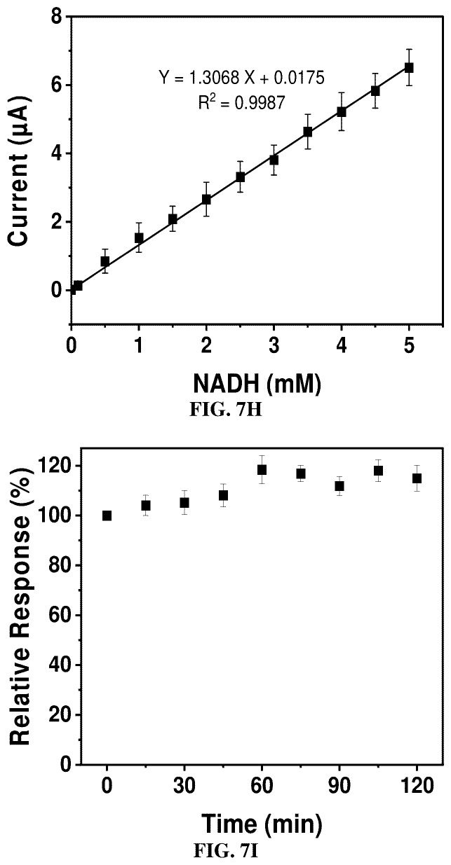

FIGS. 7A-7I show electrochemical polymerization of Toluidine blue O (TBO). FIG. 7A: washing step of TBO polymerization (FIG. 7B); stabilization step of TBO polymerization (FIG. 7C); confirming the mediator role of TBO and NADH sensing with TBO in lower potentials compared to gold electrode (FIG. 7D). Schematic showing the impact of TBO in decreasing NADH oxidation potential (FIG. 7E). Specificity of TBO modified electrode in sensing NADH compared to potential interferents (Glucose, MgCl₂, NaCl, Lactate, Sucrose, Uric acid, and Ascorbic acid) (FIG. 7F). Chronoamperometry responses of TBO modified electrode to different concentrations of NADH from 0 to 5 mM with 0.5 mM steps between each (FIG. 7G). Calibration curve of NADH (H), Stability of TBO modified Electrode in NADH sensing over time (FIG. 7I). All the figures present the mean and SD of the minimum 4 replicates.

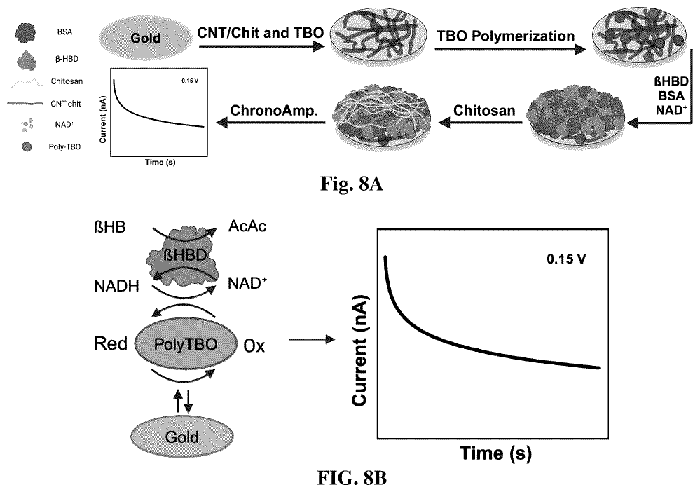

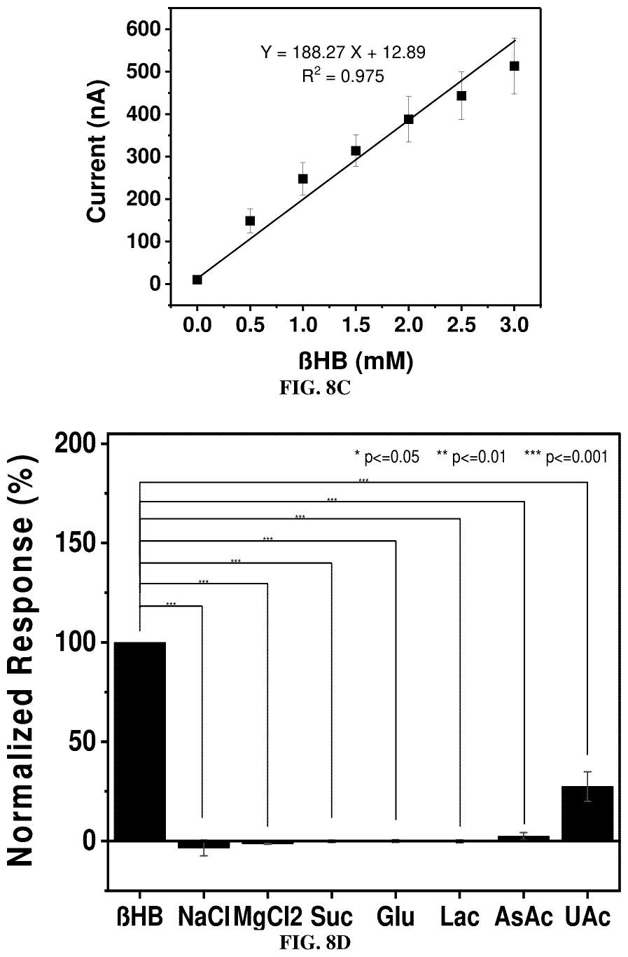

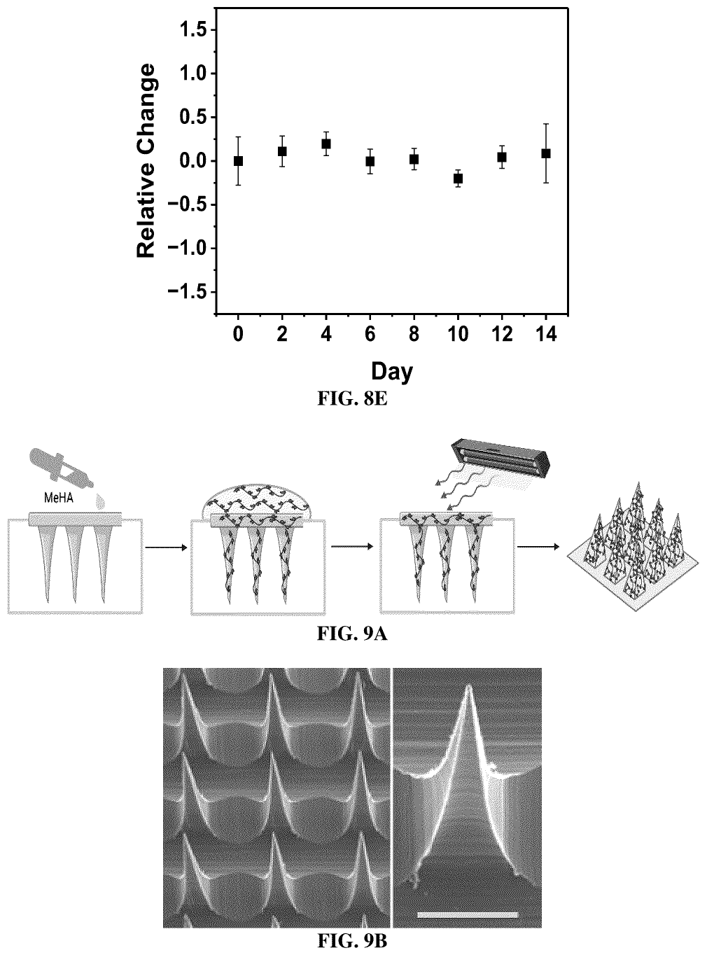

FIGS. 8A-8E show a schematic of fabrication and preparation steps of disclosed microneedle analyte sensing device (FIG. 8A). Schematic showing the β-HB sensing mechanism (FIG. 8B). Calibration curve of β-HB enzymatic biosensor (FIG. 8C). Storage stability of β-HB biosensor in the room temperature over time in the package (FIG. 8D). Specificity of ketone body biosensor to β-HB compared to other potential interferents (FIG. 8E). The figures are presenting the mean and SD of at least 4 replicates.

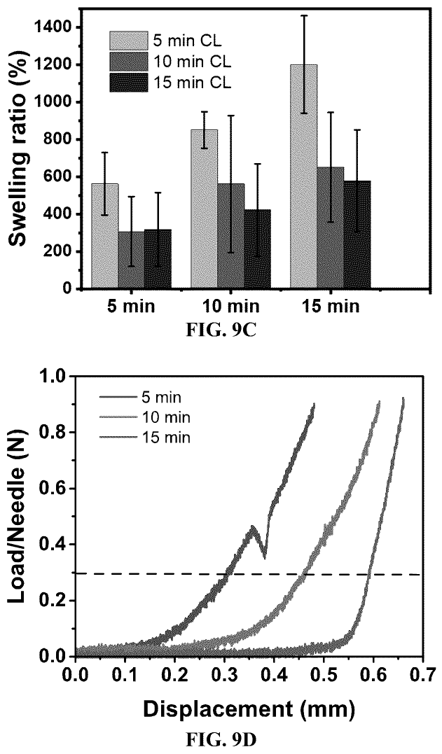

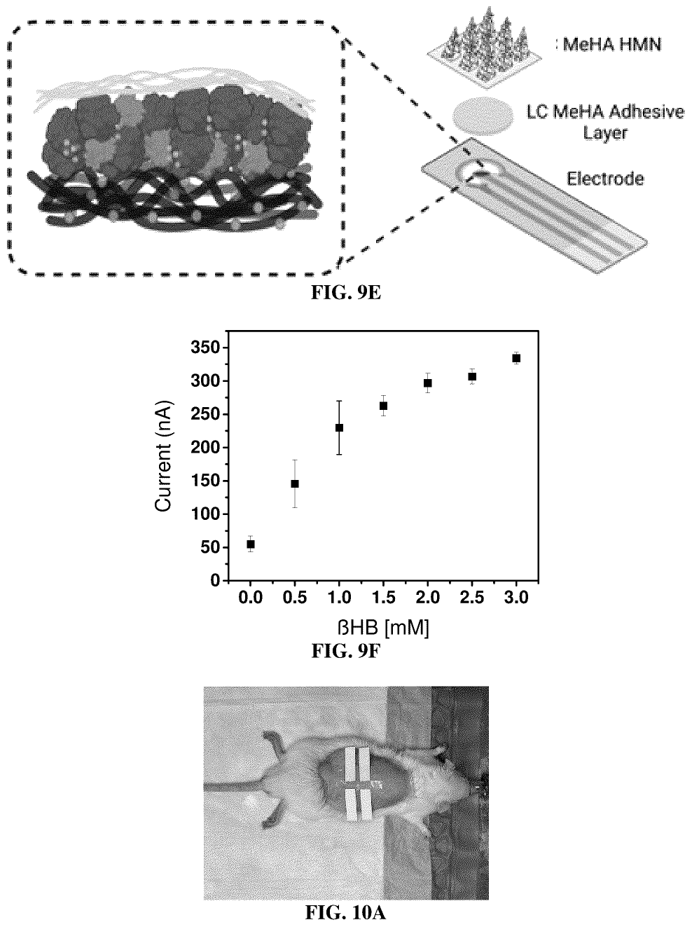

FIGS. 9A-9F show a disclosed microneedle analyte sensing device fabrication procedure. FIG. 9A: SEM images of MeHA microneedle, the scale bar is 200 μm (FIG. 9B). Swelling properties of MeHA microneedles with different crosslinking time at the different contact times (FIG. 9C). Compression mechanical properties of MeHA microneedles with different crosslinking times (FIG. 9D). Schematic showing implementation of MeHA microneedle to the ketone body biosensor (FIG. 9E). Calibration curve of Ketone body biosensor with MeHA microneedle (mean and SD of N=4).

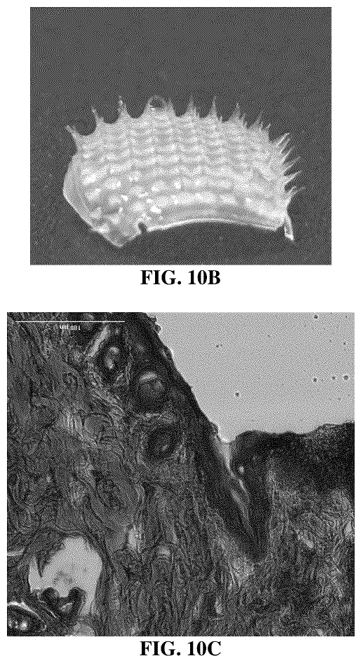

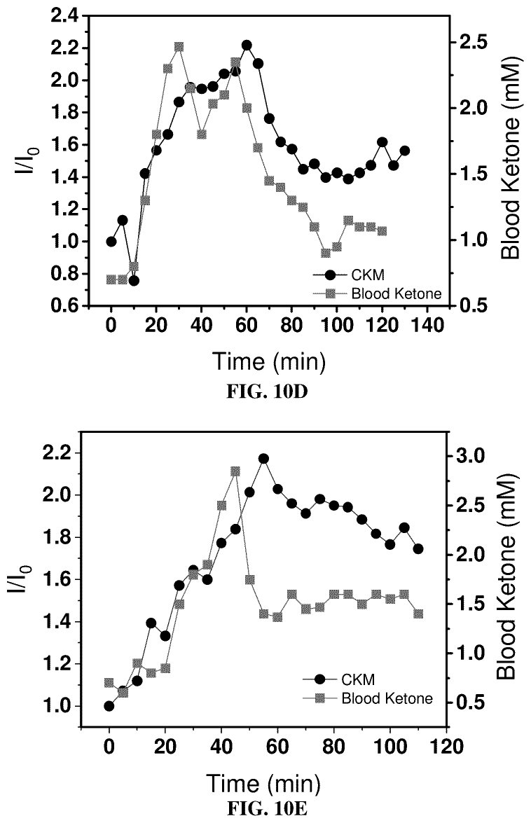

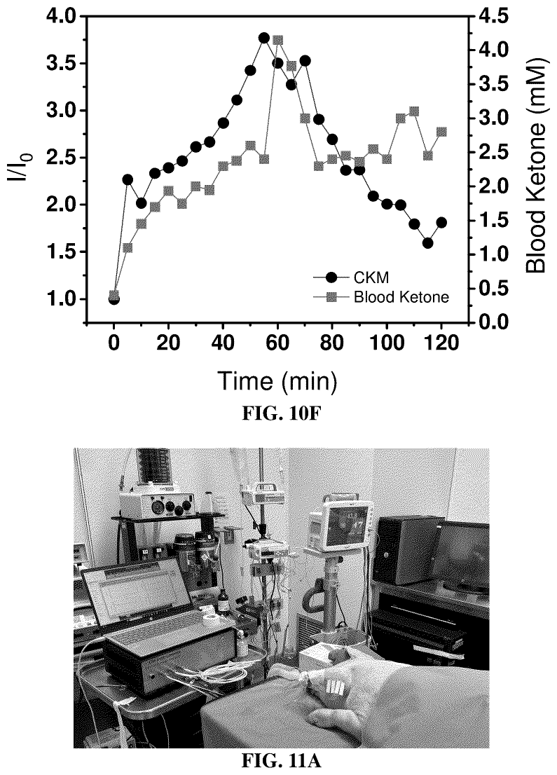

FIGS. 10A-10F show the rat under anesthesia with applied electrodes on the dorsal skin (FIG. 10A), Swelled MeHA disclosed microneedle analyte sensing device patch after in vivo study on the rat (FIG. 10B). H&E staining, histology image of rat skin approving the rupture in the stratum corneum, scale bar=300 μm (FIG. 10C). Continuous monitoring of blood ketone with developed microneedle analyte sensing device on healthy rats (FIGS. 10D, 10E, and 10F).

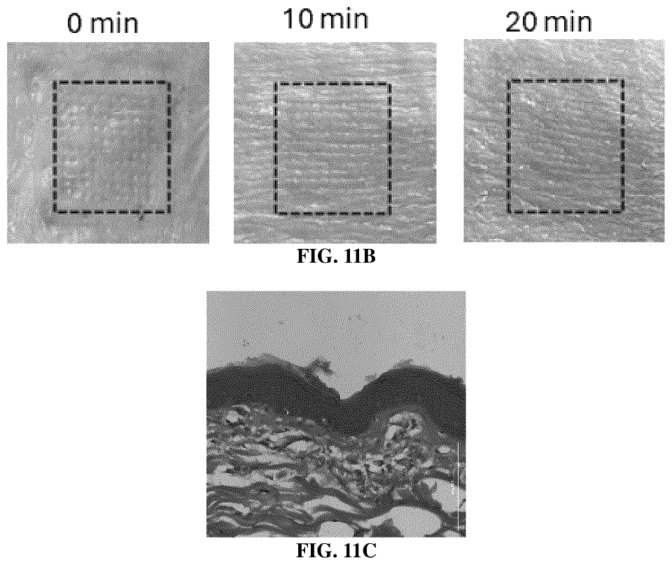

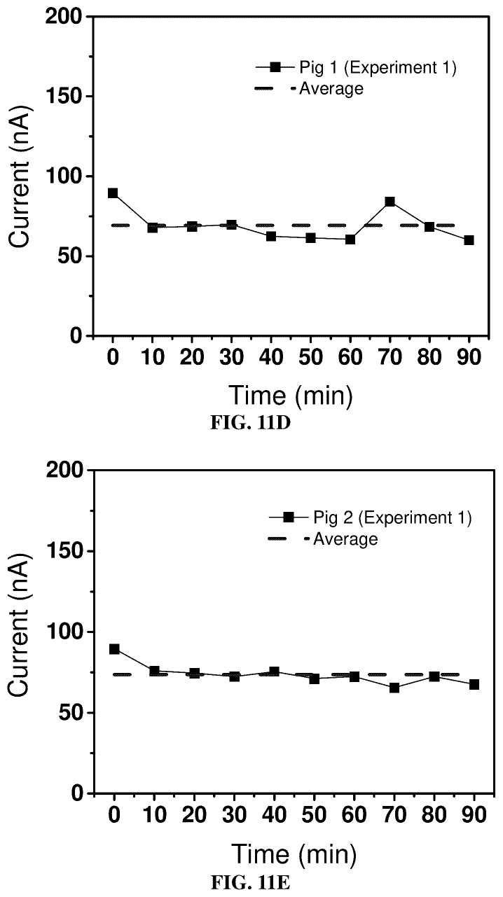

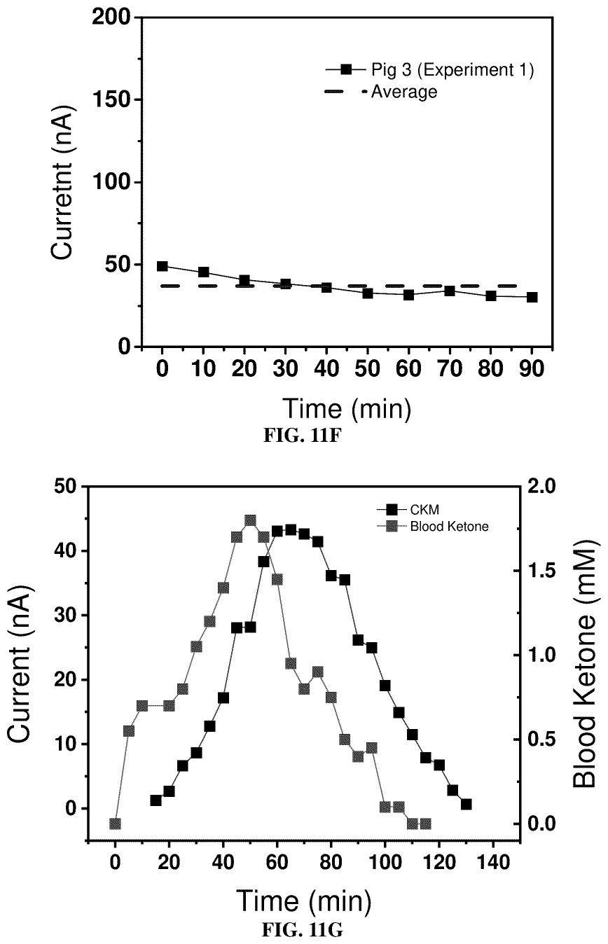

FIGS. 11A-11J show the pig live animal model in surgery room under anesthesia for ketone monitoring (FIG. 11A), Recovery of needle traces on pig skin-skin fully-recovered after 20 minutes (FIG. 11B). H&E staining histology image, showing the skin rupture induced by hydrogel microneedle, scale bar=300 μm (C). Stability of electrode responses over time on three different pigs-blood ketone level was zero during the surgery (FIGS., 11E, and 11F). Pig experiments with ketone (β-HB) infusion cross-validated with blood ketone level (FIGS. 11G, 11H, 11I, and 11J).

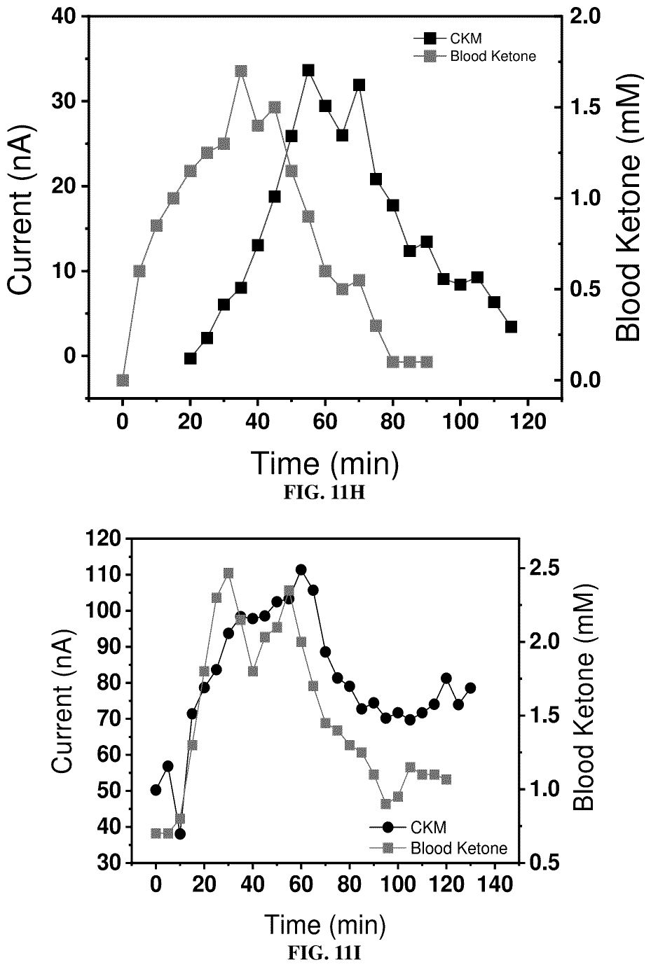

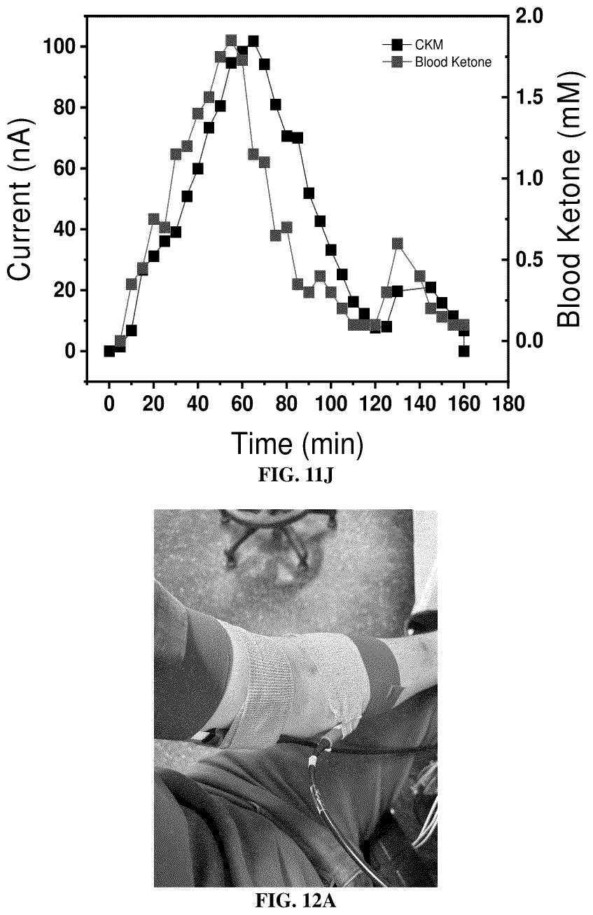

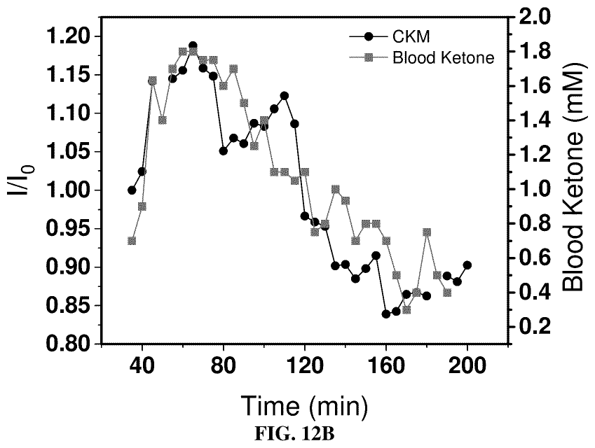

FIGS. 12A and 12B show a preliminary in-human testing of microneedle analyte sensing device. FIG. 12A: microneedle analyte sensing device was applied to the forearm of a healthy volunteer and used for continuous monitoring of ketone after consumption of ketone solution. FIG. 12B: microneedle analyte sensing device was used for continuous measurement while blood-samples were collected every 10 mins for blood-based measurement using a ketone meter for cross validation.

### DETAILED DESCRIPTION

Disclosed are methods, systems, and devices that pertain to a microneedle analyte sensing device. Techniques, systems, and devices are disclosed for the detection of analytes in living things using microneedle-based biosensors. The sensing mechanism relies on the catechol-quinone chemistry inherent to the dopamine molecules that are covalently linked to the polymer structure of the microneedle analyte sensing device patch. The dopamine serves the dual-purpose of acting as a redox mediator for measuring the byproduct of oxidation of beta-hydroxybutyrate, the primary ketone bodies, while also facilitating the formation of a crosslinked microneedle analyte sensing device patch. A universal approach involving pre-oxidation and detection of the generated catechol compounds was introduced to correlate the sensor response to the β-HB concentrations. Further demonstration confirmed that real-time tracking of a decrease in ketone levels of T1D rat model is possible using the microneedle analyte sensing device in conjunction with a data-driven machine learning model that considers potential time delays.

In an aspect, the disclosed microneedle analyte sensing device uses an array of microneedles that penetrate a surface of a biological tissue to detect changes or fluctuations in certain biomarkers in biological fluid (such as, interstitial fluid, transdermal fluid, and/or extracellular fluid). By detecting such changes or fluctuations, the devices can be used to monitor the progression of diseases and illnesses, among other conditions.

In any aspect or embodiment described herein, the disclosed microneedle analyte sensing device can be implemented by loading the microneedles with electrochemical transducers or electrodes, which can have different chemical functionalities towards biochemical and physiological analytes (e.g., ketone, acetoacetate, beta-hydroxybutyrate, lactate, acetone or glucose). In any aspect or embodiment described herein, the disclosed microneedle analyte sensing device can employ various electrochemical techniques to perform electrochemical reactions directly at the microneedle and biological fluid interface and transduce that information into an electrical signal that can be detected or measured by amperometry, impedance spectroscopy, voltammetry, and/or potentiometry in situ.

In any aspect or embodiment described herein, the disclosed microneedle analyte sensing device can be implemented transdermally by applying the device to the skin of a user. In any aspect or embodiment described herein, the disclosed microneedle analyte sensing device is integrated into a skin adhesive patch or transdermal patch, which can be applied to the skin of a user to monitor (e.g., transdermally monitor) physiological and biochemical parameters (e.g., ketone). Upon application of the patch to the skin, the microneedles penetrate the skin so that biological fluid (e.g., interstitial fluid, transdermal fluid, extracellular fluid, and/or blood) contacts the microneedle. In any aspect or embodiment described herein, the adhesive patch, or transdermal patch, can further be integrated with electronics to allow communication and signal transmission.

In any aspect or embodiment described herein, the microneedle analyte sensing device can measure the concentration of β-hydroxybutyrate, an important index of ketone bodies, directly inside the interstitial fluid of the skin. For example, in any aspect or embodiment described herein, the analyte detection and β-hydroxybutyrate level analysis are performed through electrochemical enzymatic detection of β-hydroxybutyrate by the microneedle electrodes and to facilitate β-hydroxybutyrate dehydrogenase enzyme-catalyzed oxidation of β-hydroxybutyrate to acetylacetate with the concomitant reduction of cofactor nicotinamide adenine dinucleotide (NAD+) to nicotinamide adenine dinucleotide (NADH). The disclosed microneedle analyte sensing device efficiently incorporates an electrochemical enzymatic detection system into a microneedle that penetrates the skin's surface layer, accessing the biological fluid (e.g., interstitial fluid and/or transdermal fluid) to facilitate and regulate the redox reaction. The microneedle analyte sensing device can continuously record the resulting electrical signals, which enables on-the-spot detection of diabetic biomarkers, such as ketone. The presence of the biomarker, analytes, or metabolites of interest can result in changes or perturbations in the detected current or potential.

In any aspect or embodiment described herein, the disclosed microneedle analyte sensing device can be implemented to continuously analyze and measure the concentration of β-hydroxybutyrate inside the interstitial fluid of the skin. This continuous ketone monitoring (CKM) sensor, utilizing the β-hydroxybutyrate dehydrogenase enzyme biocatalytic reaction, is achieved by overcoming key challenges related to the confinement of the enzyme/cofactor pair (HBD/NAD+) and enabling low-potential and fouling-resistant NADH oxidation. In any aspect or embodiment described herein, the cofactor is a HBD cofactor (e.g., a HBD cofactor comprising nicotinamide adenine dinucleotide (NAD+)).

In any aspect or embodiment described herein, amperometry, impedimetric biosensors, conductometry, voltammetry, and/or potentiometry can be used to detect electrical signals generated by the electrochemically mediated enzymatic reaction. For example, in any aspect or embodiment described herein, the chemical information can be converted to the electrical signals via electrochemistry, and the device can be interfaced with electronic readout. As used herein, the term “electrochemically mediated enzymatic reaction” can refer to a biochemical reaction catalyzed by an enzyme, wherein the reaction is influenced, controlled, and/or facilitated by an applied electrical potential or current. Such mediation may involve electron transfer between an electrode and the enzyme, redox cycling of a mediator, and/or modulation of reaction conditions through electrochemical means. In any aspect or embodiment described herein, the enzyme comprises β-hydroxybutyrate dehydrogenase, which catalyzes the reversible oxidation of β-hydroxybutyrate to acetoacetate. The electrochemical mediation may enhance enzymatic activity, facilitate electron transfer, and/or enable real-time monitoring of reaction progress.

The device implemented based on some embodiments of the disclosed technology can perform continuous ketone body monitoring, providing rapid diagnosis and/or treatment of the diabetic ketoacidosis.

In any aspect or embodiment described herein, the electrode structure is contained within the microneedle structure. For example, in any aspect or embodiment described herein, the electrode structure includes an electrically conductive material that is embedded within the microneedle structure. In any aspect or embodiment described herein, to enhance the electrical conductivity of the microneedle electrode, poly(3,4-ethylenedioxythiophene): polystyrene sulfonate (PEDOT:PSS), a highly conductive and biocompatible polymer, is integrated into the microneedle. In any aspect or embodiment described herein, for example, dopamine-hyaluronic acid (DA-HA) is first dissolved in a solution of PEDOT:PSS (DHP solution); and then, a mixture of HBD, tyrosinase, and NAD+ was added to the DHP solution and mixed. In any aspect or embodiment described herein, the electrically conductive material further comprises at least one of a metal nanoparticle, graphene, graphene-based material, MXene, conductive polymer, polyaniline, polypyrrole, an ionomer, carbon nano tube, or a combination thereof.

In any aspect or embodiment described therein, the disclosed microneedle analyte sensing device includes a working electrode (WE) assembly for β-hydroxybutyrate detection comprising materials that interact with β-hydroxybutyrate. For example, in any aspect or embodiment described herein, the materials include redox mediators (e.g., the redox mediators comprises at least one of dopamine, conjugated dopamine, functionalized dopamine, crosslinked dopamine, metal-complexed dopamine, phenanthroline quinones, 5,5-dihydroxy-4,4-bitryptamine, Prussian Blue, poly toluidin blue O (PTBO), toluidine blue O (TBO), or a combination thereof). For example, the redox mediator can be integrated into a material of the microneedle electrode. The working electrode of the microneedle analyte sensing device can be engineered to detect β-hydroxybutyrate utilizing dopamine acting as a mediator for a redox reaction. In any aspect or embodiment described herein, the working electrode is attached to a laser-induced graphene (LIG).

In any aspect or embodiment described herein, the redox mediator includes or is dopamine. Dopamine, as a biocompatible redox mediator, enables NADH detection at lower voltage (0.3 V instead of 1 V), providing antifouling properties. In conjunction with tyrosinase, dopamine facilitates the formation of a tightly crosslinked microneedle analyte sensing device with enhanced mechanical strength.

In any aspect or embodiment described herein, the material of the working electrode may also include at least one of β-hydroxybutyrate dehydrogenase, NAD+, and Tyrosinase, which are incorporated into the polymeric backbone of the microneedle analyte sensing device. In any aspect or embodiment described herein, the polymeric backbone of the microneedle analyte sensing device is made of hydrogel. For example, in any aspect or embodiment described herein, the hydrogel comprises, or consists of, hyaluronic acid (HA), methacrylated hyaluronic acid (MeHA), gelatin, methacrylated gelatin, alginate, methacrylated alginate, chitosan, methacrylated chitosan, collagen, methacrylated collagen, or combinations thereof. In any aspect or embodiment described herein, the microneedles of the working electrode are homogeneous throughout the microneedles.

In any aspect or embodiment described herein, the disclosed microneedle analyte sensing device includes a counter electrode. In any aspect or embodiment described herein, the counter electrode is a microneedle coated with metal nanoparticles. For example, in any aspect or embodiment described herein, the metal nanoparticles comprises, or consists of, platinum, silver, gold, palladium, or combinations thereof. In any aspect or embodiment described herein, the counter electrode is made of ultraviolet (UV)-cured epoxy (e.g., a polymer material that undergoes rapid photopolymerization upon exposure to UV light, resulting in a durable and chemically stable structure). The epoxy formulation may be optimized to provide flexibility, crack resistance, and enhanced electrochemical performance. In any aspect or embodiment described herein, the microneedles of the counter electrode are homogeneous throughout the microneedles.

In any aspect or embodiment described herein, the disclosed microneedle analyte sensing device includes a reference electrode (RE). In any aspect or embodiment described herein, the reference electrode is a microneedle coated with silver-silver chloride (Ag/AgCl). In any aspect or embodiment described herein, the reference electrode is made of ultraviolet (UV)-cured epoxy (e.g., a polymer material that undergoes rapid photopolymerization upon exposure to UV light, resulting in a durable and chemically stable structure). In any aspect or embodiment described herein, the microneedles of the reference electrode are homogeneous throughout the microneedles.

In any aspect or embodiment described herein, the disclosed microneedle analyte sensing device is connected to an electrical circuit, which processes (e.g., programmed to process) the electrical signal generated by the electrochemically mediated enzymatic reaction.

In any aspect or embodiment described herein, the disclosed microneedle analyte sensing device is fabricated by, for example, methods of cost-effective manner based on some embodiments of the disclosed technology. By way of example, FIGS. 4A-4H show exemplary fabrication and characterization of the microneedle analyte sensing device of the present disclosure. In any aspect or embodiment described herein, the microneedle analyte sensing device of the present disclosure is fabricated as shown in FIG. 7A.

In any aspect or embodiment described herein, the β-HB analysis is performed through an electrochemical mediated enzymatic reaction, relying on the HBD-catalyzed oxidation of β-HB to acetylacetate (AcAc) with the concomitant reduction of NAD+ to NADH. The electrochemically mediated enzymatic reaction with the β-HB in the biological fluid of the user can be detected to enable the in situ analyte monitoring. In any aspect or embodiment described herein, the in situ analyte monitoring means detecting and measuring analytes, which include, but are not limited to, ketones or β-hydroxybutyrate, within the biological system where they naturally occur, without the need for extraction, transport, and/or external processing.

In any aspect or embodiment described herein, the disclosed microneedle analyte sensing device is based on the use of NAD-dependent dehydrogenase type enzyme (e.g., β-hydroxybutyrate dehydrogenase). The disclosed microneedle analyte sensing device demonstrates a stable electrochemical detection of the NADH reaction product, with no apparent surface fouling.

In any aspect or embodiment described herein, the microneedle analyte sensing device of the present disclosure integrates the enzymes, the enzyme co-factor, and the redox mediators within a polymeric backbone of the microneedle, and therefore the enzymes, the enzyme co-factor, and the redox mediators are within (e.g., dispersed and/or entrapped within) the microneedle. In any aspect or embodiment described herein, the microneedle analyte sensing device can include a hydrogel as the polymeric backbone that contain, disperses, or entraps the at least one enzyme (e.g., HBD enzyme), the at least one cofactor (e.g., HBD cofactor), and/or the redox mediator within a hydrogel material.

In any aspect or embodiment described herein, the working electrode, the counter electrode, and the reference electrode are incorporated into a single hydrogel microneedle array to generate a wearable continuous ketone monitoring device (e.g., as shown FIG. 8A). The single microneedle array is designed to provide minimally invasive, pain-free insertion into the skin, facilitating direct contact with interstitial fluids for continuous monitoring. The incorporation of all three electrodes into a single, compact microneedle array reduces the device's size, improving wearability, and user comfort while ensuring reliable and consistent performance for continuous monitoring of ketone levels in various settings.

For example, an aspect of the present disclosure provides a microneedle analyte sensing device for continuously monitoring and measuring an analyte in a biological fluid of a user, the microneedle analyte sensing device comprising: a plurality of microneedles operable to penetrate a surface of a biological tissue of the user and contact the plurality of microneedles with the biological fluid when the microneedle analyte sensing device is attached to the surface of the biological tissue, wherein: at least one microneedle of the plurality of microneedles is a working electrode that detects an electrical signal generated from an electrochemically mediated enzymatic reaction with the analyte in the biological fluid of the user; at least one microneedle of the plurality of microneedles is a counter electrode; and at least one microneedle of the plurality of microneedles is a reference electrode.

In any aspect or embodiment described herein, the at least one microneedle that is the working electrode is made of a hydrogel. In any aspect or embodiment described herein, the at least one microneedle that is the counter electrode is made of ultraviolet (UV)-cured epoxy and coated with metal nanoparticles, wherein the metal nanoparticles comprise platinum, silver, gold, palladium, or combinations thereof. In any aspect or embodiment described herein, the at least one microneedle that is the reference electrode is made of ultraviolet (UV)-cured epoxy and coated with silver-silver chloride (Ag/AgCl).

In any aspect or embodiment described herein, the hydrogel comprises at least one of hyaluronic acid, methacrylated hyaluronic acid, gelatin, methacrylated gelatin, alginate, methacrylated alginate, chitosan, methacrylated chitosan, collagen, methacrylated collagen, or a combination thereof. In any aspect or embodiment described herein, the at least one microneedle that is the working electrode comprises enzymes integrated in the hydrogel, and wherein the enzymes comprise at least one of beta-hydroxybutyrate dehydrogenase (HBD), tyrosinase, or a combination thereof. In any aspect or embodiment described herein, the at least one microneedle that is the working electrode comprises a HBD cofactor integrated in the hydrogel, wherein the HBD cofactor comprises nicotinamide adenine dinucleotide (NAD+). In any aspect or embodiment described herein, the at least one microneedle that is the working electrode comprises a redox mediator integrated in the hydrogel to facilitate electron transfer in the electrochemically mediated enzymatic reaction. In any aspect or embodiment described herein, the at least one microneedle that is the working electrode comprises an electrically conductive material integrated in the hydrogel to increase electrical conductivity of the at least one microneedle.

In any aspect or embodiment described herein, the microneedle analyte sensing device detects the electrical signal generated from the electrochemically mediated enzymatic reaction in situ.

In any aspect or embodiment described herein, the analyte is ketone, acetoacetate, beta-hydroxybutyrate, lactate, acetone, or glucose.

In any aspect or embodiment described herein, the biological fluid is at least one of interstitial fluid, transdermal fluid, extracellular fluid, blood, or a combination thereof.

In any aspect or embodiment described herein, the plurality of microneedles are disposed on a substrate or within a substrate that the plurality of microneedles are operable to penetrate. In any aspect or embodiment described herein, the microneedle analyte sensing device is integrated into a transdermal patch. In any aspect or embodiment described herein, the biological tissue is skin or stratum corneum (e.g., skin or stratum corneum of the user).

In any aspect or embodiment described herein, the redox mediator comprises at least one of dopamine, conjugated dopamine, functionalized dopamine, crosslinked dopamine, metal-complexed dopamine, poly toluidine blue O (PTBO), toluidine blue O (TBO), or combinations thereof. In any aspect or embodiment described herein, the electrically conductive material comprises at least one of poly(3,4-ethylenedioxythiophene): polystyrene sulfonate (PEDOT:PSS), a metal nanoparticle, graphene, MXene, conductive polymer, polyaniline, polypyrrole, ionomer, carbon nano tube, or a combination thereof. In any aspect or embodiment described herein, the electrochemically mediated enzymatic reaction is detectable using at least one of amperometry, impedimetry, conductometry, voltammetry, potentiometry, or a combination thereof.

In any aspect or embodiment described herein, the microneedle analyte sensing device is a continuous ketone monitoring (CKM) sensor.

By way of further example, an aspect of the present disclosure provides an analyte sensing device comprising: the microneedle analyte sensing device of the present disclosure; and an electrical circuit electrically comprising a data processing unit, wherein: the electrical circuit is connected to the microneedle analyte sensing device and processes (e.g., programmed to process) the electrical signal generated by the electrochemically mediated enzymatic reaction; and the data processing unit comprises a processor and a memory, and processes (e.g., configured to process) the electrical signal as data representative of one or more parameters of the analyte.

In any aspect or embodiment described herein, the analyst sensing device further comprises a wireless communication unit in communication with the electrical circuit to transmit a processed signal to a user interface. In any aspect or embodiment described herein, the user interface comprises a smartphone, a personal computer, a laptop, a tablet, a wearable device, a smart home device, an Internet of Things (IoT) device, or a combination thereof.

In any aspect or embodiment described herein, the analyte sensing device is a continuous ketone monitoring (CKM) sensor.

In any aspect or embodiment described herein, the microneedle analyte sensing device may be manufacturing by a method comprising: applying a first composition to an electrode, wherein the first composition comprises a redox mediator, chitosan, and an electrically conductive material; curing the first composition to generate a first layer; applying a second composition to the first layer to generate a second layer on top of the first layer, wherein the second composition comprises at least one enzyme and at least one enzyme cofactor; and applying a third composition to the second layer to generate a third layer on top of the second layer, wherein the third composition comprises chitosan. In any aspect or embodiment described herein, the method further comprises preparing the first composition by mixing the redox mediator, the chitosan, and the electrically conductive material. In any aspect or embodiment described herein, the method further comprises preparing the second composition by mixing the at least one enzyme and the at least one enzyme cofactor. In any aspect or embodiment described herein, the electrode is a screen printed gold electrode. In any aspect or embodiment described herein, the electrically conductive material comprises or is carbon nanotubes (e.g., multi-walled carbon nanotubes, single-walled carbon nanotubes, double-walled carbon nanotubes, or a combination thereof). In any aspect or embodiment described herein, curing the first composition via cyclic voltammetry to generate a first layer. In any aspect or embodiment described herein, the at least one enzyme comprise at least one of beta-hydroxybutyrate dehydrogenase (HBD), tyrosinase, or a combination thereof; the at least one enzyme cofactor comprises nicotinamide adenine dinucleotide (NAD+). In any aspect or embodiment described herein, the redox mediator comprises at least one of dopamine, conjugated dopamine, functionalized dopamine, crosslinked dopamine, metal-complexed dopamine, poly toluidin blue O (PTBO), toluidine blue O (TBO), or combinations thereof.

In any aspect or embodiment described herein, the microneedle analyte sensing device may be manufacturing by a method comprising: applying or injecting a composition for a working- electrode to a micro-mold for microneedles of the working electrode, wherein the composition for the working-electrode comprises, consists essentially of, or consists of, a hydrogel, at least one enzyme, at least one enzyme cofactor, a redox mediator, and an electrically conductive material. In any aspect or embodiment described herein, the method further comprises preparing the composition for the working-electrode by mixing a hydrogel, at least one enzyme, at least one enzyme cofactor, a redox mediator, and an electrically conductive material.

In any aspect or embodiment described herein, the method further comprises preparing a counter electrode, comprising applying or injecting ultraviolet (UV)-curable epoxy to a micro-mold for microneedles of the counter electrode; curing the UV-curable epoxy of the counter electrode, optionally via ultraviolet light; and coating the counter electrode with metal nanoparticles, wherein the metal nanoparticles comprise platinum, silver, gold, palladium, or combinations thereof.

In any aspect or embodiment described herein, the method further comprises preparing a reference electrode, comprising applying or injecting ultraviolet (UV)-curable epoxy to a micro-mold for microneedles of the reference electrode; curing the UV-curable epoxy of the reference electrode, optionally via ultraviolet; and coating the reference electrode with silver-silver chloride (Ag/AgCl).

In any aspect or embodiment described herein, the hydrogel comprises at least one of hyaluronic acid, methacrylated hyaluronic acid, gelatin, methacrylated gelatin, alginate, methacrylated alginate, chitosan, methacrylated chitosan, collagen, methacrylated collagen, or combinations thereof. In any aspect or embodiment described herein, the at least one enzyme comprise at least one of beta-hydroxybutyrate dehydrogenase (HBD), tyrosinase, or a combination thereof. In any aspect or embodiment described herein, the at least one enzyme cofactor comprises nicotinamide adenine dinucleotide (NAD+). In any aspect or embodiment described herein, the redox mediator comprises dopamine, conjugated dopamine, functionalized dopamine, crosslinked dopamine, metal-complexed dopamine, poly toluidin blue O (PTBO), toluidine blue O (TBO), or combinations thereof. In any aspect or embodiment described herein, the electrically conductive material comprises at least one of poly(3,4-ethylenedioxythiophene): polystyrene sulfonate (PEDOT:PSS), a metal nanoparticle, graphene, MXene, conductive polymer, polyaniline, polypyrrole, ionomer, carbon nano tube, or a combination thereof.

In any aspect or embodiment described herein, the microneedles of the working electrode are homogeneous throughout the microneedles. In any aspect or embodiment described herein, the microneedles of the counter electrode are homogeneous throughout the microneedles. In any aspect or embodiment described herein, the microneedles of the reference electrode are homogeneous throughout the microneedles. In any aspect or embodiment described herein, the method further comprises forming one or more molds corresponding to shapes of (i) the microneedles of the working electrode, (ii) the microneedles of the counter electrode, (iii) the microneedles of the reference electrode, or (iv) a combination thereof.

In any aspect or embodiment described herein, the disclosed microneedle analyte sensing device is connected to an electrical circuit which processes (e.g., programmed to process) the electrical signal generated by the electrochemically mediated enzymatic reaction. The electrical circuit comprises a data processing unit in communication with a signal processing circuit, and the data processing unit comprises a processor and a memory and is configured to process the electrical signal as data representative of one or more parameters of the analytes. In certain embodiments, the data processing unit and the signal processing circuit may be implemented using a computing device such as a smartphone, personal computer, laptop, tablet, server, embedded system, microcontroller, single-board computer, wearable device (e.g., smartwatch, smart glasses, fitness tracker), edge computing device, industrial computer, cloud-based computing platform, virtual machine, networked computing system, programmable logic device, automotive control system, robotics control unit, smart home device, Internet of Things (IoT) device, medical device (e.g., diagnostic machine, infusion pump, pacemaker), or any other suitable electronic device capable of processing signals and data.

In any aspect or embodiment described herein, the disclosed microneedle analyte sensing device or analyte sensing device comprises a wireless communication unit in communication with the electrical circuit to transmit the processed signal. For example, in any aspect or embodiment described herein, the wireless communication unit may include, but is not limited to, Bluetooth, Wireless Fidelity (Wi-Fi), Fifth Generation (5G) mobile networks, Fourth Generation Long-Term Evolution (4G LTE) mobile networks, Near Field Communication (NFC), Zigbee, Z-Wave, Long Range (LoRa) wireless technology, Sigfox, Radio Frequency Identification (RFID), Ultra-Wideband (UWB), satellite communication, infrared communication, any other suitable wireless communication technology, or a combination thereof.

In any aspect or embodiment described herein, a microneedle analyte sensing device or analyte sensing device is used to detect, measure or monitor a biomarker or an analyte in a biological fluid of a user via a three-step process (as shown in FIG. 3D). Step 1: pre-oxidation step at 0.3 V to convert catechol to quinone. To ensure enough supply of quinones, an initial pre-oxidation step was performed by applying a positive potential to convert the catechol to quinone. Step 2: 10-s wait time to allow quinone-NADH interaction. Step 3: detection step at 0.3 V to detect the amount of catechol produced. The catechol production and quinone consumption can be used to monitor β-HB.

In any aspect or embodiment described herein, an agarose gel may be used as phantom gel skin-mimicking model.

In any aspect or embodiment described herein, a microneedle analyte sensing device, analyte sensing device, or microneedle patch, is fabricated with a micro-molding technique. In any aspect or embodiment described herein, the microneedle analyte sensing device or analyte sensing device is fabricated using a micro-molding technique, wherein a high-precision master mold is first created using methods like photolithography or laser ablation. Biocompatible materials, such as polymers or hydrogels, are then poured or injected into the mold. The material is solidified through curing or drying to ensure proper needle formation. Once hardened, the microneedle analyte sensing device is removed from the mold to preserve its structural integrity. Additional processing, such as surface coating with drugs or functionalization, may be applied to enhance performance.

In any aspect or embodiment described herein, a microneedle analyte sensing device or analyte sensing device is fabricated with photolithography and etching involving patterning a photosensitive material followed by chemical or plasma etching to create precise silicon or metallic microneedles. In any aspect or embodiment described herein, a microneedle analyte sensing device or analyte sensing device is fabricated with laser micromachining that uses laser ablation to cut or shape microneedles from metal, polymer, or ceramic substrates. In any aspect or embodiment described herein, a microneedle analyte sensing device or analyte sensing device is fabricated with 3D printing or additive manufacturing. In any aspect or embodiment described herein, a microneedle analyte sensing device or analyte sensing device is fabricated with electrochemical deposition, such as electroforming. In any aspect or embodiment described herein, a microneedle analyte sensing device or analyte sensing device is fabricated with lithography.

For successful skin penetration, the sharpness and mechanical strength of microneedle are critical. In any aspect or embodiment described herein, a microneedle analyte sensing device is fabricated by combining HBD, tyrosinase, NAD⁺, and DHP solution with DA-HA hydrogel possessing 10% DA (HMN (10%)) or 17% DA (HMN (17%)). In any aspect or embodiment described herein, “DHP-tyrosinase” produces a mechanically strong patch. Considering the physical properties and mechanical strength of the patches, a DA conjugation rate of 17% was selected for the fabrication of the HMN-WE of microneedle analyte sensing device. For example, in any aspect or embodiment described herein, the DHP solution comprising DA-HA hydrogel comprising about 15% to about 20% DA (e.g., about 15% to about 20%, about 15% to about 19%, about 15% to about 18%, about 15% to about 17%, about 15% to about 16%, about 16% to about 20%, about 16% to about 19%, about 16% to about 18%, about 16% to about 17%, about 17% to about 20%, about 17% to about 19%, about 17% to about 18%, about 18% to about 20%, about 18% to about 19%, about 19% to about 20%, about 15%, about 16%, about 17%, about 18%, about 19%, or about 20%).

An important characteristic of a wearable biosensor is its ability for continuous, long-term, on-body monitoring, relying on limited release of sensing components. Thus, ensuring efficient entrapment/dispersion/distribution within the crosslinked hydrogel network is crucial. HBD and NAD+ leaching by fabricating a microneedle analyte sensing device with and without tyrosinase on agarose hydrogel (mimicking dermis with ISF) covered with a parafilm layer (representing the water-impermeable stratum corneum and epidermis) to mimic the skin's stratum corneum were examined. The highly crosslinked microneedle analyte sensing device also minimized the leaching of the sensing components within the polymeric network, which enhanced the stability of the sensor during prolonged measurements. This is particularly vital for dehydrogenase-based enzymatic sensors that require additional redox mediators and co-factors.

In any aspect or embodiment described herein, the disclosed microneedle analyte sensing device can be implemented as wearable minimally-invasive system for continuous monitoring of diabetic ketoacidosis, which requires prompt medical attention once it occurs.

In any aspect or embodiment described herein, the disclosed microneedle analyte sensing device is portable and powered by a battery, allowing for convenient and uninterrupted operation. In any aspect or embodiment described herein, the battery comprises or is a rechargeable lithium-ion, lithium-polymer, nickel-metal hydride, or any other suitable energy storage technology. In any aspect or embodiment described herein, the device may include power management features (e.g., energy-efficient circuitry, low-power operation modes, automatic sleep/wake functions, adaptive power consumption based on real-time usage, or a combination thereof). Additionally, in any aspect or embodiment described herein, the device may support wireless charging, fast-charging capabilities, or energy harvesting from body movement, ambient light, or thermal gradients to extend battery life, or a combination thereof. The portable design may enable users to wear, carry, or attach the device to clothing or accessories, ensuring continuous monitoring in various environments, including home, workplace, or clinical settings.

In any aspect or embodiment described herein, the disclosed microneedle analyte sensing device or analyte sensing device allows continuous ketone monitoring for 7 to 14 days without the need for replacement or interruption. In any aspect or embodiment described herein, the disclosed microneedle analyte sensing device or analyte sensing device allows continuous ketone monitoring for 10 to 14 days without the need for replacement or interruption. In any aspect or embodiment described herein, the disclosed microneedle analyte sensing device allows continuous ketone monitoring for 1 to 30 days without the need for replacement or interruption.

In any aspect or embodiment described herein, the disclosed microneedle analyte sensing device provides (e.g., configured to provide) pain-free biosensing and is a highly integrated biocompatible devices, these devices can be fabricated on an industrial scale and at low cost. The disclosed microneedle analyte sensing device or analyte sensing device can perform monitoring and biosensing applications without involving fluid sampling or extraction. For example, a feature of the microneedle analyte sensing device or analyte sensing device is that the extraction of bodily fluids (e.g., transdermal fluid) is not required. Through the execution of electrochemistry at the microneedle-transdermal fluid interface, chemical information can be extracted and directly transduced to the electrical circuits.

In any aspect or embodiment described herein, the disclosed microneedle analyte sensing device or analyte sensing device is implemented to support personalized medicine by continuously measuring ketone levels in a user's body, integrated into a wearable device such as a patch, wristband, or smartwatch. In any aspect or embodiment described herein, the microneedle analyte sensing device further includes a processor configured to analyze the real-time ketone data in conjunction with additional health metrics, such as glucose levels, heart rate, and physical activity, to provide personalized health insights. The system generates alerts when ketone levels fall outside predefined thresholds, allowing users to make timely adjustments to diet, medication, or lifestyle. In any aspect or embodiment described herein, a user interface, user-friendly mobile application, or dashboard displays historical trends and actionable recommendations, enhancing the user's ability to manage conditions such as diabetes, or metabolic disorders. In any aspect or embodiment described herein, the user interface, user-friendly mobile application, or dashboard includes smartphone, personal computer, laptop, tablet, wearable device, smart home device, Internet of Things (IOT) device, or a combination thereof.

In any aspect or embodiment described herein, the term “electrical stimulus signal” refers to an applied electrical input, including but not limited to voltage, current, or a combination thereof, that is used to initiate, modulate, or sustain an electrochemically mediated enzymatic reaction within a microneedle analyte sensing device. In any aspect or embodiment described herein, the electrical stimulus signal may be direct current (DC) or alternating current (AC) and may be applied in various forms, such as constant potential, pulsed potential, cyclic voltammetry, or chronoamperometry, depending on the operational requirements of the device. In any aspect or embodiment described herein, the electrical stimulus signal facilitates electron transfer between an electrode and an enzyme, such as β-hydroxybutyrate dehydrogenase, to drive the oxidation or reduction of an analyte, thereby enabling real-time monitoring or detection of target molecules, including β-hydroxybutyrate.

As used herein, the term “counter electrode (CE)” refers to an electrode that functions as a reference point for completing the electrical circuit in an electrochemical system. The counter electrode provides a pathway for electron flow to balance the current generated at the working electrode during an electrochemically mediated enzymatic reaction. In any aspect or embodiment described herein, the counter electrode is made of a conductive material (e.g., platinum, carbon, gold, or a combination thereof) and is positioned to ensure efficient charge transfer within the system.

As used herein, the term “reference electrode (RE)” refers to an electrode that provides a stable and known potential against which the potential of the working electrode is measured in an electrochemical system. The reference electrode maintains a constant electrochemical potential, independent of the reaction occurring at the working electrode, ensuring accurate and reproducible measurements. In any aspect or embodiment described herein, the reference electrode comprises a material, such as, silver/silver chloride (Ag/AgCl) and is in contact with a biological fluid that stabilizes its potential.

In another aspect, disclosed herein is a method for measuring an analyte within a biological fluid of a user. The method includes providing a microneedle analyte sensing device, wherein the microneedle analyte sensing device comprises a plurality of microneedles operable to penetrate a surface of a biological tissue of the user and contact the plurality of microneedles with the biological fluid when the microneedle analyte sensing device is attached to the surface of the biological tissue (e.g., skin or stratum corneum); placing the microneedle analyte sensing device on the surface of the biological tissue of the user to contact (e.g., transdermally contact) the plurality of microneedles with the biological fluid; applying an electrical stimulus signal to at least one microneedle of the plurality of microneedles; detecting an electrical signal arising by an electrochemically mediated enzymatic reaction with the analyte in the biological fluid of the user exposed to the at least one microneedle; and determining a concentration of the analyte based on the electrical signal.

In any aspect or embodiment described herein, at least one microneedle of the plurality of microneedles is a working electrode that detects the electrical signal generated from the electrochemically mediated enzymatic reaction with the analyte in the biological fluid of the user. In any aspect or embodiment described herein, at least one microneedle of the plurality of microneedles is a counter electrode. In any aspect or embodiment described herein, at least one microneedle of the plurality of microneedles is a reference electrode.

In any aspect or embodiment described herein, the plurality of microneedles are disposed on a substrate or within a substrate that the plurality of microneedles are operable to penetrate. In any aspect or embodiment described herein, the microneedle analyte sensing device is integrated into a transdermal patch. In any aspect or embodiment described herein, the biological tissue is skin or stratum corneum. In any aspect or embodiment described herein, the electrical signal is transferred through the at least one microneedle to an electrical circuit.

In any aspect or embodiment described herein, the method further includes sending the electrical signal from the electrical circuit to a data processing unit. For example, in any aspect or embodiment described herein, the data processing unit (i) comprises a processor and a memory, and/or (ii) processes (e.g., configured to process) the electrical signal as data representative of one or more parameters of the analytes. In any aspect or embodiment described herein, the method further includes sending the electrical signal from the data processing unit to a wireless communication unit in communication with the electrical circuit to transmit a processed signal to a user interface. For example, in any aspect or embodiment described herein, the user interface comprises a smartphone, a personal computer, a laptop, a tablet, a wearable device, a smart home device, a Internet of Things (IoT) device, or a combination thereof.

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where a combination is disclosed, it is understood that each possible sub-combination of the elements of that combination is also disclosed. Conversely, where different elements or groups of elements are individually disclosed, combinations thereof are also disclosed.

Where elements are presented in list format or as alternative members of a group (e.g., a Markush group), it is understood that each possible subgroup of the elements is also disclosed, and any one or more elements can be removed from the list or group.

Where a range of numerical values is recited, it is understood that the endpoints and each intervening integer value and each fraction thereof, as well as each subrange, between the recited endpoints (upper and lower limits) of that range are specifically disclosed. The endpoints of all ranges are included within the range and are independently combinable. Where a value has an inherent limit, that inherent limit is specifically disclosed. Where a value is explicitly recited, it is understood that values which are about the same as the recited value are specifically disclosed.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether or not the specific exclusion is recited in the specification.

It is further understood that the present disclosure encompasses analogs, derivatives, prodrugs, metabolites, salts, solvates, hydrates, clathrates and polymorphs of all the compounds/substances disclosed herein, as appropriate. The specific recitation of “analogs”, “derivatives”, “prodrugs”, “metabolites”, “salts”, “solvates”, “hydrates”, “clathrates” or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned or shown without recitation of any of these forms, unless stated otherwise or the context clearly indicates otherwise.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated by reference herein in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by those of ordinary skill in the art to which this application belongs.

As used in the specification and the claims, the indefinite articles “a” and “an” and the definite article “the” can include plural referents as well as singular referents unless specifically stated otherwise or the context clearly indicates otherwise.

The terms “or/and” and “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, that is, “one or more” of the elements so conjoined. Other elements may optionally be present other than the elements specifically identified by the “and/or” or “or/and” clause, whether related or unrelated to those elements specifically identified. Thus, as not limiting examples, the phrase “A or/and B” means “either A or B, or both A and B”, and the phrase “A, B or/and C” means “either A, B or C, or any combination or all thereof”.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (that is, “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used in the specification and the claims, all transitional terms such as “comprising”, “containing”, “having”, “including”, “possessing”, “holding”, “carrying”, “bearing”, “composed of”, “characterized by” and the like are open-ended and inclusive, that is, mean including but not limited to and do not exclude additional, unrecited element(s) or method step(s). It is expressly contemplated that all embodiments, and claims reciting one of the open-ended transitional phrases can be written with any other transitional phrase, which may be more limiting, unless clearly precluded by the context or art. Only the transitional term “consisting of” is closed, that is, excludes any additional, unrecited element or method step, and the transitional term “consisting essentially of” is semi-closed, that is, only allows inclusion of additional, unrecited element(s) or method step(s) that do not materially affect the basic and novel characteristic(s) of that particular embodiment.

The term “exemplary” as used herein means “serving as an example, instance or illustration”. Any embodiment or feature characterized herein as “exemplary” should not be construed as preferred or advantageous over other embodiments or features.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

In some embodiments, the term “substantially all” means at least about 90%, 95%, 96%, 97%, 98% or 99%. In some embodiments, the term “substantially free” means no more than about 10%, 5%, 4%, 3%, 2% or 1% by weight or molarity, or no more than about 1000 ppm, 500 ppm, 400 ppm, 300 ppm, 200 ppm or 100 ppm.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The term “pharmaceutically acceptable” means that a substance (e.g., an active ingredient or an excipient) is generally safe, non-toxic and suitable for use in contact with the cells, tissues and organs of a subject without excessive irritation, allergic response, immunogenicity and other adverse reaction. A “pharmaceutically acceptable” excipient or carrier of a pharmaceutical composition is also compatible with the other ingredients of the composition.

The terms “treat”, “treating” and “treatment” include alleviating, ameliorating, reducing the incidence, frequency or severity of, slowing or stopping the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk or likelihood of developing, and delaying the onset of a medical condition or one or more symptoms or complications associated with the condition.

The term “medical conditions” (or “conditions” for brevity) includes diseases and disorders. The terms “diseases” and “disorders” are used interchangeably herein.

The term “user” refers to an animal, including but not limited to a mammal, such as a primate (e.g., a human, a chimpanzee or a monkey), a rodent (e.g., a rat, a mouse, a guinea pig, a gerbil or a hamster), a lagomorph (e.g., a rabbit), a bovine (e.g., a cattle), a suid (e.g., a pig), a caprine (e.g., a sheep), an equine (e.g., a horse), a canine (e.g., a dog) or a feline (e.g., a cat). The terms “subject”, “user” and “patient” may be used interchangeably herein in reference to a subject/patient (e.g., a mammalian subject/patient such as a human subject/patient) having a medical condition.

The term “compound” or the like (e.g., “molecule”) encompasses salts, solvates, hydrates, clathrates and polymorphs of that compound or a salt of that compound. A “solvate” of a compound comprises a stoichiometric or non-stoichiometric amount of a solvent molecule (e.g., water, acetone or an alcohol [e.g., ethanol]) bound non-covalently to the compound. A “hydrate” of a compound comprises a stoichiometric or non-stoichiometric amount of water molecule bound non-covalently to the compound. A “clathrate” of a compound contains molecules of a substance (e.g., a solvent) enclosed in a crystal structure of the compound. A “polymorph” of a compound is a crystalline form of the compound. The specific recitation of “salt”, “solvate”, “hydrate”, “clathrate” or “polymorph” with respect to a compound or a group of compounds in certain instances of the disclosure shall not be interpreted as an intended omission of any of these forms in other instances of the disclosure where the term “compound” or the like (e.g., “molecule”) is used, or where the compound or the group of compounds is mentioned or shown, without recitation of any of these forms, unless stated otherwise or the context clearly indicates otherwise.

### General Methods

Materials: The pharma-grade sodium hyaluronate (HA, MW 300 kDa) was purchased from Bloomage Co. Ltd (China). PEDOT:PSS (PH 1000-Hareaus Clevios) was purchased from Ossila. Dopamine hydrochloride (DA), N-(3 Dimethylaminoropyl)-N′-ethylcarbodiimide (EDC), N-Hydroxysuccinimide (NHS), dialysis tubing cellulose membrane (MWCO=14000), ethylene glycol (EG), β-Nicotinamide adenine dinucleotide (NADH), B-Nicotinamide adenine dinucleotide hydrate (NAD+), DL-β-Hydroxybutyric acid sodium salt (β-HB), β-Hydroxybutyrate Dehydrogenase from Pseudomonas lemoignei (HBD), Tyrosinase from mushroom, Insulin human, Streptozotocin, Potassium ferricyanide (III), Silver/Silver Chloride (60/40) paste for screen printing, UV curable resin, sodium L-lactate, uric acid, ascorbic acid, and glucose were purchased from Sigma Aldrich. Conductive Silver Epoxy was purchased from MG Chemicals. 10×10 microneedle molds were purchased from Micropoint Technologies Pte Ltd. 10× phosphate buffer saline (PBS) was purchased from BioShop. Standard gold (2 mm WE) electrodes were purchased from CH Instruments (CHI). Gold (2 mm WE) phase zero sensors were purchased from Conductive Technologies Inc (CTI). Kapton® Polyimide Film was purchased from Mcmaster-Carr. Tegaderm was purchased from 3M. Porcine ears were purchased from a local supermarket.

Dopamine mediated NADH sensing: A solution of 10 mM dopamine was prepared in PBS (pH=7.4), and the pre-oxidation step was carried out by doing a positive scan between −0.2 to 0.6 V on CHI electrodes using the CHI 1040C potentiostat; followed by NADH addition to the dopamine solution. The solution was mixed for 10 s, and CV was performed in the potential range of −0.2 to 0.6 V. These steps were repeated for different concentrations (0 to 8 mM) of NADH. The NADH CV was conducted between −1.3 to 1.3 V in PBS (pH=7.4). The scan rate was 0.05 V s⁻¹. CHI electrodes were cleaned in H₂SO₄ (0.5 M) for 30 cycles between −0.4 to 0.6 V with a 0.05 V s⁻¹ scan rate. CTI electrodes were cleaned in H₂SO₄ (0.1 M) and cycled for 10 cycles between 0 to 1.5 V at 0.1 V s⁻¹. All the potentials were measured versus Ag/AgCl reference electrode.

Dopamine mediated β-HB sensing: An enzyme solution composed of HBD (0.5 U μL-1), NAD⁺ (50 mM), and 10× concentrated solution of β-HB was allowed to react for 5 minutes at room temperature. A pre-oxidation positive scan between −0.2 to 0.6 V was conducted in dopamine solution (11 mM in PBS pH 7.4) on CTI electrodes (PalmSense4). The enzyme solution was added to the dopamine solution with a ratio of 1:10, and a CV scan was conducted between −0.2 to 0.6 V. The scan rate was 0.05 V s⁻¹. To study the importance of sensing components, the same experiment was carried out in the absence of each of the components namely, DA, HBD, and NAD+ at 0 and β-HB (10 mM). Also, for the specificity study, the same experimental setup was followed with the difference that the β-HB was replaced with ascorbic acid (0.2 mM), uric acid (0.2 mM), lactate (5 mM), and glucose (2.5-15 mM). The 10× concentrated stock of uric acid was prepared in PBS pH 8. The catechol oxidation and quinone reduction normalized difference was calculated following equation 1. CTI electrodes were cleaned in H₂SO₄ (0.1 M) and cycled for 10 cycles between 0 to 1.5 V at 0.1 V s⁻¹.

$$Normalized Difference = \frac{\begin{matrix}( Peak current of interfering analyte - \\ peak current at 0 mM β - HB )\end{matrix}}{peak current at 0 mM β - HB} \qquad ( 1 )$$

Sensing mechanism validation: A solution containing dopamine (10 mM) and NADH (0, 1, or 2.5 mM) in PBS (pH=7.4) was drop-casted on the CTI electrodes. A positive SWV scan was conducted between −0.2 to 0.6 V for the pre-oxidation step (PalmSense4). The waiting times of 5, 10, and 15 s were applied before the detection SWV scan which was performed between −0.2 to 0.6 V. The scan rate was 0.05 V s⁻¹. CTI electrodes were cleaned in H₂SO₄ (0.1 M) and cycled for 10 cycles between 0 to 1.5 V at 0.1 V s⁻¹.

Dopamine-hyaluronic acid (DA-HA) synthesis and characterization: DA-HA synthesis and characterization was adapted from previous work. HA (2 g) was dissolved overnight in Milli-Q water (200 mL). The next day, DA was conjugated through EDC-NHS coupling by the addition of EDC (485 mg), NHS (291.5 mg), and DA (0.5 g). The pH was lowered to 5 by the addition of hydrochloric acid (1M). The solution was stirred overnight in the dark. The DA-HA solution undergoes a 3-day dialysis process using cellulose membrane dialysis tube in deionized water. The water changed daily and kept stirring throughout the process. After dialysis the DA-HA was freeze dried. The DA-HA conjugation rate was analyzed using 1H-NMR and UV-Vis spectroscopy (BioTek, Synergy H1).

Microneedle patch fabrication: MN patches were prepared with the micro-molding technique. To prepare HMN-WE patches, 17% DAHA (50 mg) was dissolved in a solution containing 5 wt % PEDOT:PSS, ethylene glycol, and PBS (pH=6); then, a mixture of HBD (0.1 U mg⁻¹ DAHA), tyrosinase (200 U mg⁻¹ DAHA), and NAD⁺ (500 mM) were added to the as-prepared DA-HA solution and mixed thoroughly. The polymeric mixture starts to crosslink upon the addition of tyrosinase but the tyrosinase activity is not at its optimum at pH 6. After NaOH (10 μL) was added, the mixture's pH increased to 7, increasing the tyrosinase activity and accelerating crosslinking in situ. The prepared mixture was transferred to the molds and centrifuged for 5 minutes at 7800 rpm and kept in the desiccator for 3 hours. Finally, the HMN patches were kept at room temperature overnight to be completely dried and removed from the molds. The reference and counter MN electrodes were prepared with UV-cured epoxy. The UV-cured epoxy was drop casted onto MN molds and subjected to multiple vacuum cycles to remove bubbles, and then were placed under UV for 3 minutes to cure. The reference electrodes were prepared with a layer of thin Ag/AgCl coating and left to dry at room temperature overnight. The gold on the counter electrodes were E-beam evaporated with chrome (40 nm) and gold (200 nm). A silver wire was connected to the MN electrodes with silver epoxy. Laser-induced graphene (LIG) was prepared using the Boss Laser LS1416.

β-HBD enzyme in combination with a redox polymer (poly toluidine blue O) is employed for-HB detection. Poly toluidin blue O (PTBO) is used as a redox mediator to reduce the overpotential associated with NADH and to minimize the potential interference from the oxidation of different molecules such as ascorbic acid, glucose, uric acid, and others commonly present in the body. As shown in FIG. 7A, Toluidine blue O (TBO) in the monomer form was mixed with carbon nanotube (CNTs) and drop-casted on the screen-printed gold electrodes. Having dried, the electrodes were immersed in 5 mM TBO solution (pH=4, PB 0.1 M), and cyclic voltammetry was performed between −0.4 to 1 V (vs. Ag/AgCl) with a scan rate of 50 (mV/s) to polymerize the TBO monomers, followed by 5 more cyclic voltammetry cycles in the same potential range in the PB 0.1 M (pH=4). This step which uniquely developed to facilitates washing the monomers while enhancing the polymerization. Then, a new round of cyclic voltammetry in PBS 0.01 M (pH=7.4) was performed as another washing step and to adjust the pH of the electrode. This step is also uniquely included to minimize the effect of pH change on enzyme stability. Last, a proper amount of NAD+ solution (18.75 mM) was drop-casted on the working electrode to extract any remaining trace of TBO monomer for minimizing the risk of leaching. The use of the poly toluidine blue O (PTBO) combined with carbon nanotubes (CNTs) and cyclic voltammetry polymerization methods contributes to a highly stable and conductive sensing interface. Additionally, separating the sensor from the interstitial fluid collector, such as the hydrogel microneedle patches, enhances sensor stability by minimizing interference from body fluids and ensuring long-term performance. This separation reduces the risk of degradation or leaching of the polymer and enzyme components, maintaining the sensor's reliability during continuous monitoring.

Microneedle analyte sensing device physical characterization: The structure of HMNs and the porosity of hydrogels were inspected by scanning electron microscopy (SEM) technique (HITACHI SU 5000 FESEM). First, gold (2 nm) was deposited on the HMN patches and thin films. For the porosity experiment, thin films of different compositions were prepared, and immersed in DI water for 12 min; then, immediately soaked in liquid nitrogen to stabilize the pores and then were cut into small pieces and freeze-dried for 72 hours. Additionally, mechanical strength testing was carried out by Instron 5548 micro tester. Different HMN patches, namely DHP-tyrosinase, DHP+tyrosinase, HMN 17%, and HMN 10% were trimmed around the needle array border and mounted on an anvil set to a final height of 100 mm; then, a gradually increasing load up to 10 N was applied to the HMN patches and force per needle was plotted versus the displacement. The swelling rate of DHP-tyrosinase, DHP+tyrosinase, and HMN 17% was studied on 1.4 wt % agarose gel. After trimming, the HMN patches were applied on the agarose gel for 2 hours, and the swelling rate was calculated using equation 2:

$$S welling Rate = \frac{Swelled State Weight - Dried State Weight}{Dried State Weight} \qquad ( 2 )$$

With the same token, the swelling rate of HMN 17% patch was investigated at different time points (10, 30, 60, and 90 minutes) to determine the swelling profile of the HMN patch.

The electrical conductivity of the HMN 17% at both dried and swelled states were measured using four-point probe method (Keithley 4200-SCS). The resistivity of the HMN patches was first measured; then, the resistivity correction factor of 4.532 was used for calculating the resistance. Considering the resistance and height of needles, the conductivity of the HMN patches was calculated using equation 3:

$$Conductivity = \frac{1}{Thickness \times Resistance} \qquad ( 3 )$$

Ex vivo validation of microneedle analyte sensing device: Thawed porcine skins were cleaned in DI water and then immersed in various β-HB solutions in PB (0.1 M pH 7.4) overnight at 4° C. Prior to microneedle analyte sensing device patch application, the porcine skin was compressed in paper towels to remove the excess solution. After patch application, the LIG electrodes were placed over and held together with Tegaderm. The MN-RE and MN-CE were then applied and taped down with Tegaderm. For β-HB calibration, a pre-oxidation step was conducted at a 0.3 V applied potential for 20 s (CHI 1040C). After 10 s waiting time, a detection step was conducted at a 0.3 V applied potential for 50 s. Due to the swelling and deformation of the microneedle analyte sensing device after application on the porcine skin, the sensor is only used once. The same experimental protocol was conducted to study the effect of pH using porcine skins equilibrated in 0 and 1.5 mM PB (pH 7.0 and 7.3). For the stability experiment, the same experimental protocol was implemented in β-HB (0.75 mM) and data was collected every 10 minutes for 2 hours.

Characterization of the sensing component leaching: Enzymes and NAD⁺ release experiments were performed on 1.4 wt % agarose gel covered with a thin layer of parafilm, mimicking the stratum skin layer. After trimming, the HMN 17% patches were applied on agarose gel and were kept for on and 10 hours, then the released amounts of NAD⁺ and enzymes were calculated. To measure the released NAD+, the agarose gel was dissolved by heat, and UV absorbance was read at 265 nm (BioTek, Synergy H1). To eliminate any possible interference from PEDOT:PSS presence, it was not included in the patch fabrication. Enzyme release was measured by Bradford assay; due to the interference that NAD⁺ and PEDOT:PSS presence can add to the Bradford assay, they were excluded from patch fabrication for the enzyme release experiment. After the agar application, HMN patches were removed and placed in DI water for 24 hours to release the remaining enzymes in the patches; the release components in solution were then measured with Bradford assay. The subtraction of the measured protein in solution from the initial loaded enzyme in the HMN patch determines the released enzyme into the agarose gel.

Microneedle analyte sensing device mechanical stability and adhesion: Mechanical stability tests were performed on porcine skin equilibrated win 1.5 mM β-HB in PB (0.1 M pH 7.4) overnight at 4° C. Prior to microneedle analyte sensing device patch application, the porcine skin was compressed in paper towels to remove the excess solution. After HMN-WE, MN-RE, and MN-CE were applied and taped down with Tegaderm, 100 consecutive cycles of twisting and bending were conducted. Electrochemical measurements were conducted (PalmSense4) when the porcine skin was at normal, bent, and twisted states. The chronoamperometry measurement was conducted as follows: 1) a pre-oxidation step at a 0.3 V applied potential for 20 s, 2) 10 s waiting time, and 3) a detection step was conducted at a 0.3 V applied potential for 50 s.

To evaluate adhesiveness of microneedle analyte sensing device to the skin, a lap shear test was performed. An HMN-WE was applied onto a porcine skin that was fixed on the stiff paperboard using a superglue and taped down with Tegaderm. After 50 minutes of swelling the sample was mounted on the Universal Testing Machine (Test Resources 120R) and pulled at a rate of 5 mm/min.

In vivo validation of microneedle analyte sensing device: Animal studies were conducted in accordance with the Animal Welfare Act Regulations; all protocols were approved by the University of Waterloo's Institutional Animal Care and Use Committee. An established streptozotocin (STZ)-induced diabetic rat (T1D) was used to evaluate the microneedle analyte sensing device performance. Male Sprague Dawley rats (Charles River, 100-150 gr) were injected with (65 mg kg⁻¹ i.p) which causes the degradation of the rat's pancreatic beta-cells secretion activity. The STZ rat's glucose and ketone levels were monitored with commercial glucose (Contour Next ONE meter, Ascensia, Inc. USA) and ketone (Freestyle Libre, Abbott Inc.) meter. Rats with blood ketone levels above 0.6 mM were selected for this study. Rats were fasted on the day of the experiment for 4-5 hours. The rats were placed under anesthesia with isoflurane gas at 5% for 1 minute and then 2.5% for the remaining duration of experiment. The rats' backs were shaven with a shaver and hair removal cream. The HMN-WE patches were applied and allowed to reach maximum swelling for about 60 minutes. The MN-CE, MN-RE, HMN-WE were then taped down with Tegaderm. After approximately 60 minutes, insulin (10U) was injected into the rat subcutaneously. Chronoamperometry measurements were conducted at 0.3 V with a pre-oxidation step for 20 seconds and detection step for 50 seconds (CHI 1040C). Chronoamperometry measurements were conducted every 5 minutes. The reference blood ketone levels from the rats' tails were measured every 10 minutes using the Freestyle Libre ketone meter.

Statistical analysis: All statistical analyses were conducted using Origin2023b. The statistical difference between groups in the specificity test was analyzed using Dunnett's one-way ANOVA (p=0.05) and shown in GP style (0.1234 (ns), 0.0332 (*), 0.0021 (**), 0.0002 (***), p<0.0001 (****)) to compare the interfering analytes against β-HB. Each experiment for the different interfering analytes was conducted on three different sensors. In the swelling experiment, the statistical difference between the DHP-tyrosinase, DHP+tyrosinase, and HMN (17%) was analyzed using Dunnett's one-way ANOVA (p=0.05) and shown in GP style (0.1234 (ns), 0.0332 (*), 0.0021 (**), 0.0002 (***), p <0.0001 (****)). The statistical difference between the sensor response in pH 7.0 and 7.3 was analyzed using two-way ANOVA (p=0.05) and shown in GP style (0.1234 (ns), 0.0332 (*), 0.0021 (**), 0.0002 (***), p <0.0001 (****)). Linear regression analysis fitting with a 95% confidence interval (CI) and scale error with reduced chi-square was applied on the calibration curves for the validation of dopamine mediated sensing, including the catechol oxidation and quinone reduction NADH interaction calibration curves and quinone β-HB calibration curve. The linear regression analysis was also applied on the ex vivo calibration curve. The sensitivity was extracted from the slope of the curve. The Michaelis-Menten fitting with a 95% CI and scale error with reduced chi-square was applied on the catechol oxidation calibration experiments. The sensitivity was extracted from the slope of the linear curve.

The sensor response was calculated based on the catechol recovery ratio using the below equation:

$$Catechol Recovery = \frac{{I}_{Detection}}{{I}_{Pre - oxidation}} \qquad ( 4 )$$

The I_{Pre-oxidation} current corresponds to all detectable catechol in the system while the I_{Detection} current represents the amount of catechol recovered after NADH reacts with quinone.

A normalization technique was employed in the chronoamperometry data to better illustrate the relationship between the pre-oxidation and detection chronoamperometry curves in response to different concentrations of β-HB. To this end, the pre-oxidation and detection chronoamperometry curves were multiplied using the normalization factor shown below:

$$Normalization factor ( x mM ) = \frac{pre - oxidation current ( 0 mM )}{pre - oxidation current ( X mM )} \qquad ( 5 )$$

All data is shown as mean±standard deviation. Sample sizes are indicated in the figure captions.

In in vivo experiments, to remove the impact of noise in the microneedle outputs, a moving average filter was applied to every detection and pre-oxidation current profiles across all microneedles tested. Then, a linear fit with a 95% confidence interval was applied on the detection-pre-oxidation ratio as a function of the time of experiments. With a decreasing trend over time, the points within the confidence band were passed forward, and the ones outside were removed. Following this, a stochastic gradient boosting decision tree (GBDT) framework was used to predict the ketone levels. GBDT is an ensemble ML model that constructs a series of decision trees in a stage-wise fashion as ‘weak learners’ where each subsequent tree improves over the errors of its preceding tree. Each tree was trained on a random subset of the training datapoints, which incorporates randomness and prevents overfitting of the model. The model considered three input features—the two endpoints of the filtered pre-oxidation and detection currents, and the inverse of the corresponding time of the experiment. The model performance was evaluated using three error metrics-MARD, MAD, and number of mistakes in binary classification of ketone levels above or below 1.5 mM to determine if the ML framework can distinguish between high/low ketone levels. Following previous reports, the accuracy for ketone levels ≥1.5 mM was calculated as percentage whereas ketone levels <1.5 mM was calculated in molarity since small differences can cause a large percent change. The delay in sensor response compared to blood ketone was determined by shifting the sensor output forward in time with varying minutes. The optimal delay was the number of minutes that gave minimum MAD, MARD, and mistakes in binary classification.

Electrode Preparation: Carboxylated multi-walled carbon nanotubes (0.7%) were immersed in 0.5 wt % chitosan solution (dissolved in 0.1M acetic acid), and sonicated for 10 hrs to become well dispersed; then, 0.5 mM of Toluidine Blue-O (TBO) solution was prepared with dispersed multi-walled carbon nanotubules (MWCNTs). The screen-printed electrodes were washed with ethanol, isopropanol, and milliQ water. Then, electrodes were cleaned with 10 cycles of cyclic voltammetry (CV) in 0.5 M sulfuric acid in 0-1.5 V potential window with a scan rate of 100 mV/s, and rinsed with milliQ water. This step is followed by drop-casting the 0.5 mM TBO/MWCNTs solution on the screen-printed electrodes. After drying, the electrodes were immersed in 5 mM TBO solution (pH=4, PB=0.1M), and CV was performed in −0.4 to 1 V potential window for 5 cycles at 50 mV/s; then, the electrode moved to PB solution (0.1 M, pH=7.4) and 5 more CV cycles were performed in the same potential range. Lastly, the electrodes were stabilized in PBS (0.01 M, pH=7.4) with 5 cycles of CVs between −0.4 and 0.4 V.

NADH Interaction TBO Redox Mediator: To confirm that TBO serves the function of the redox mediator in NADH sensing, first 1 mM of NADH in PBS (0.01 M, pH=7.4) was added to the electrode surface, followed by conducting CV between −0.6 to 1.2 V at the scan rate of 50 (mV/s). The oxidation peak of NADH was observed at 1 V. In another set of an experiment, the PBS (0.01M, pH=7.4) was introduced to the poly-TBO modified electrode, and then CV was performed within the potential window of −0.6 to 0.4 V. Next, 1 mM of NADH solution was added to the TBO modified electrode, and CV was performed similar to the previous procedure. An increase in 0.15 V oxidation peak was observed, highlighting the interaction between TBO and NADH.

NADH Calibration: The calibration curve of NADH was prepared using different concentrations of NADH dissolved in PBS. To this end, first PBS was added to the electrode and three consecutive chronoamperometry measurements were performed at 0.15 V. Then PBS was removed and replaced with different concentrations of NADH. The current level at 40 sec was selected as the main response.

Specificity of TBO-modified Electrode to NADH: The TBO-modified electrode must be specific to NADH to ensure it can detect NADH with high accuracy. To evaluate the specificity of NADH sensor, its response to 3 mM NADH was compared with those of different potential interferents in their physiological level viz glucose (5 mM), uric acid (0.2 mM), ascorbic acid (0.2 mM), lactate (5 mM), MgC12 (150 mM), sucrose (5 mM) as well as NaCl (150 mM). The electrode response to NADH was set as 100%, and those of the others were reported accordingly.

Enzyme Immobilization: A mixture of 0.5 ug β-HBD enzyme with 18 mM of its cofactor (NAD+) was prepared in 0.1 M Tris buffer containing BSA and drop-casted on the electrode surface, and allowed to dry. Then, 2 μL of chitosan solution (0.5 wt %) added to the enzyme layer to restrain the bioreceptor in the vicinity of the electrode surface.

Immobilized Enzyme Characterization: We evaluated the response of ketone biosensor to various concentrations of β-HB by preparing and applying β-HB solution at different concentrations (0.01 M PBS, pH=7.4) to the ketone biosensor. The electrode responses were evaluated up to 3 mM of NADH, which is in the range of interest for ketone monitoring. To this end, the chronoamperometry (0.15 V) reading at t=60 s was considered as the biosensor's response to β-HB. Additionally, the specificity of the fabricated electrode was challenged in the presence of different interferents in their physiological level, such as glucose (5 mM), lactate (5 mM), sucrose (5 mM), uric acid (0.2 mM), ascorbic acid (0.2 mM), MgCl2 (150 mM), and NaCl (150mM). The electrode response to 3 mM of-HB was assumed as 100%, and the interferents' responses were adjusted to β-HB response accordingly. Also, the storage stability of the electrodes was assessed. The electrodes were prepared and were kept in an Aluminium foil laminated polymer sealed with the hot-press method. The package layers are composed of Polyamide (JIS Z1714), Adhesive (Polyester-polyurethane), Aluminum foil, Adhesive (Urethane-free Adhesive), and Polypropylene to prevent oxygen diffusion as well as minimizing any negative effect caused by light exposure. The electrodes were packaged and stored at ambient conditions for different days, and their activity was measured against 1 mM of β-HB.

Methacrylated Hyaluronic Acid (MeHA) Hydrogel Microneedle: Methacrylic anhydrose moieties were linked to the hyaluronic acid (HA) backbone through alcoholic hydroxyl group found in the HA structure under alkaline environment. The synthesized MeHA was precipitated with acetone and further extracted with ethanol. Subsequently, extracted MeHA was redissolved in MilliQ water, and dialyzed against MilliQ water for 5 days, and later lyophilized for 3 days. Hydrogel microneedles were prepared using the micromolding technique. To this end, 50 mg of MeHA hydrogel were mixed with 2 mg of MBA (N,N′-Methylenebisacrylamide) and 2 mg of photoinitiator (IRGACURE 2959) and dissolved in 1 mL of MilliQ water. The prepared hydrogels were transferred to the microneedle molds, followed by degassing in a vacuum

environment for 5 minutes and left to dry at room temperature [10]. Before use, the microneedles were crosslinked for 15 minutes under UV exposure. Then, the microneedle patches were taken out of the molds and attached to the electrode surface using low-crosslinked (LC) MeHA hydrogel.

The surface morphology and the structure of the MeHA microneedle were inspected by FESEM (HITACHI SU 5000). Initially, 4 nm of gold nanoparticles were deposited on the microneedle patch, and then imaging was carried out at 15 kV accelerating voltage. Also, the effect of MeHA crosslinking time on its physical properties was examined. To this end, MeHA microneedles were prepared and crosslinked for 5, 10, and 15 minutes; then, they were placed on the agarose gel (1.4 wt %) covered by a thin layer of parafilm, mimicking the skin environment. Swelling rate of the MeHA microneedles was measured at 5, 10, and 15 minutes of contact with the agarose gel. In addition, the mechanical properties of MeHA microneedle subjected to different crosslinking times (i.e. 5, 10, and 15 min) were assessed using the Universal Testing Machine (Test Resources 120R). The load was applied on the MeHA microneedle patch and its displacement was recorded during the load increase.

After checking mechanical properties, MeHA patch was attached to the electrode surface using Low-Crosslinked MeHA, and a calibration curve was derived for electrodes with microneedle, while a calibration curve was previously derived for the electrodes in the absence of microneedle.

In Vivo Studies-Rat: Animal studies were conducted under the Animal Welfare Act Regulations; all protocols were approved by the University of Waterloo's Institutional Animal Care and Use Committee. Male Sprague Dawley rats were purchased from Envigo RMS (Canada) limited, and they were used while they were between 4 to 8 weeks old. To this end, rats were anesthetized using 5% isoflurane/oxygen mixture, and maintained under anesthesia using 1.5-2% isoflurane/oxygen mixture. Then, the dorsal skin of the rat was shaved with a trimmer and hair removal cream was applied to remove the remaining hairs. This step followed by patch application on the rat skin and fixed by Tegaderm tape. There is a 40-50 minutes of warm up and swelling step. Having reached the swollen state, 5 consecutive chronoamperometry measurements at 0.2 V were performed. Then, β-HB with the concentration of 1.5 M started to be infused intravenously through the tail at the rate of 1 mL/hr using a syringe pump to administer the β-HB with higher control and avoid any problem that can be raised from the sudden injection. The ketone level in ISF was monitored by CKM using chronoamperometry technique at 0.2 V every 5 minutes; blood samples were taken from the rat's tail and its ketone level was measured with the Freestyle Libre Ketone meter. After being done, the rat was recovered and returned to the cage.

In Vivo Studies-Swine: Pig in vivo studies were performed in agreement with Animal Welfare Act Regulations; all protocols were approved by the University of Waterloo's Institutional Animal Care and Use Committee as well as University Health Network (UHN, Toronto). This project is peer-reviewed for scientific merit by the Canadian Council on Animal Care (CCAC). Healthy Yorkshire pigs (110 days old) were hosted at UHN, and used in our studies. The pigs were sedated and immobilized with Atropine (0.04 mg/kg BM), Ketamine (20 mg/kg BM), and Midazolam (0.3 mg/kg BM) through intramuscular injection. Once lateral recumbency is achieved, general anesthesia was induced with 5% (isoflurane/oxygen) mixture, and maintained with 2-3% (isoflurane/oxygen) mixture provided by nose cone, followed by endotracheal intubation and peripheral catheter placement.

Following this, the skin on the pig's ear and leg were shaved, treated with hair removal cream, cleaned and dried. Then, microneedle patches were applied on the ears, and allowed them to get swollen and be warmed up for 50 minutes. This step followed by administration of 1.5 M of β-HB at the rate of 60 mL/hr using a syringe pump. The electrochemical readings were conducted every 5 minutes with the chronoamperometry technique at 0.2 V; simultaneously, blood samples were taken from the leg, and its ketone level was measured with the Freestyle Libra ketone meter.

H&E staining: The dorsal skin of the rat and the ear skin of the pig were shaved using a trimmer, and hair removal cream was applied. The skin was cleaned and microneedle patches were applied on the skin. Having euthanized, the skin parts containing the MeHA microneedle patch were collected, and after washing with 0.9% saline, the samples were fixed in neutral buffered 10% formalin for 24 h. The fixed skin samples were then cryopreserved in a 15% sucrose/PBS solution overnight at 4° C. Afterward, the samples were mounted on cork using OCT (TissueTek) and sliced into 10 μm-thick slides at −20° C. These skin slides were stained with hematoxylin and eosin for visualization of histological morphology. Staining involved immersing slides in Harris-modified hematoxylin for 30 s, followed by washing in distilled water and counterstaining with 1% eosin Y for 2 min. Subsequently, the slides underwent dehydration in 75% and 95% ethanol, followed by clearing in xylene. Images were captured at 20× magnification using a Cytation-5 multimode imager (Agilent) and processed using Gen5 software (Agilent). To observe the healing process, HMN-miR sensors were applied to shaved rat dorsal skin for 20 min, and the micropore array generated by the HMN was imaged at 0, 5, 10, and 15 min following patch removal.

### EXAMPLES

The following examples are intended only to illustrate the disclosure. Other synthetic processes, assays, studies, protocols, procedures, methodologies, techniques, reagents and conditions may alternatively be used as appropriate.

### Example 1. Microneedle Analyte Sensing Device Sensing Strategy

The microneedle analyte sensing device is a three microneedle (MN) electrode system comprised of an hydrogen microneedle (HMN) working electrode (HMN-WE), a gold-coated MN counter electrode (MN-CE), and an Ag/AgCl-coated MN reference electrode (MN-RE) where each electrode contains a 10×10 array of 850 μm needles (FIG. 1A). The HMN-WE was fabricated by mixing the dopamine (DA)-hyaluronic acid (HA) polymer with poly(3,4-ethylenedioxythiophene): polystyrene sulfonate (PEDOT:PSS), β-hydroxybutyrate dehydrogenase (HBD) enzyme, tyrosinase enzyme, and nicotinamide adenine dinucleotide, oxidized form (NAD+), as a cofactor. In this design, DA, which also serves as the redox mediator, was covalently immobilized on the HA polymer backbone of the sensor. The DA conjugation was confirmed via 1H-NMR, and its degree of conjugation was quantified using the UV-Vis absorbance method. Upon the addition of tyrosinase to an aqueous DA-HA solution, the DA-DA crosslinking in situ rapidly increased the solution viscosity (FIG. 1B). Increasing tyrosinase concentrations in a DA-HA solution resulted in an increase in UV absorbance peaks corresponding to quinone, while the catechol peak decreased. This observation demonstrates the generation of quinone moieties through catechol oxidation, leading to hydrogel crosslinking in situ. Tyrosinase can produce highly crosslinked DA-HA under physiological pH conditions, whereas other DA crosslinking approaches require high concentrations of oxidizing agents (e.g., NaIO4) or very high pH, which can adversely affect the HA and enzymes. To detect DL-β-Hydroxybutyric acid sodium salt (β-HB), HBD enzyme and NAD+ were integrated within the quinone enriched patch. The HBD catalyzes the β-HB oxidation in the presence NAD+ to produce NADH. The generated NADH oxidizes back to NAD+ by reacting with quinone to produce catechol that can be electrochemically measured on the surface of the conductive microneedle analyte sensing device electrodes and is correlated with β-HB concentration (FIG. 1C).

### Example 2. Validation of Dopamine-Mediated Sensing Mechanism

The microneedle analyte sensing device uses DA as a redox mediator to detect NADH (FIG. 2A). In this system, the quinone moieties of DA can be reduced to catechol concurrently with the oxidation of NADH to NAD+; thereby enabling the detection of β-HB. Dopamine's function as a redox mediator in NADH sensing was verified through the decrease of NADH's oxidation potential from 1 to 0.4 V when DA was present, (FIG. 2B).

To further study DA-mediated NADH sensing, a pre-oxidation step was performed in a 10 mM DA solution to convert the catechol into quinone moieties, and then NADH was added to the solution (FIG. 2C). A linear correlation between peak current and NADH was observed for both catechol oxidation and quinone reduction (FIGS. 2D and 2E). Increasing the NADH concentration resulted in an increase in the catechol oxidation peak because the quinone generated in the pre-oxidation step is subsequently converted back to catechol through its reaction with NADH (FIGS. 2C and 2D). Conversely, by increasing the NADH concentration, the quinones produced from the catechol oxidation step are consumed by reacting with available NADH prior to their reduction. Therefore, a decrease in the quinone reduction peak was observed with increasing NADH concentration (FIGS. 2C and 2E). The DA's catechol and quinone sensitivity to NADH sensing were 3.03 μA mM−1 and 1.9 μA mM−1, respectively. Next, the use of DA's catechol and quinone was examined to measure β-HB (FIGS. 2F-2H). Following the DA mediated NADH sensing experiments, cyclic voltammetry was conducted in a solution of DA, HBD, NAD+, and various concentrations of β-HB. The catechol oxidation peak current increases with increasing β-HB concentration and reaches a plateau at approximately 5 mM β-HB, congruent with Michaelis Menten kinetics as expected in enzymatic systems (FIG. 2G) with a sensitivity of 1.2 μA mM−1 within the range of 0-2 mM. On the other hand, the decrease in the quinone reduction peak current exhibited linearity with increasing β-HB concentration with a sensitivity of 0.4 μA mM−1(FIG. 2H). Even though both the catechol production and quinone consumption can be used to monitor β-HB, the catechol production exhibited a higher sensitivity and thus is preferred to establish a β-HB sensor.

Next, the impact of each sensing component (i.e., HBD, NAD+, and DA) on β-HB detection was evaluated. FIGS. 2I-2K illustrates the detection of 0 and 10 mM β-HB in the absence of DA, NAD+, or HBD. The absence of DA showed no redox peaks as the β-HB concentration increased from 0-10 mM (FIG. 2I). A negligible change in the catechol and quinone redox peaks was observed without the NAD+ cofactor, suggesting the need for NAD+ within the vicinity of the HBD for β-HB detection (FIG. 2J). The absence of HBD also does not show any changes in catechol and quinone redox peaks upon the addition of 10 mM β-HB (FIG. 2K). Next, the selectivity of DA mediated chemistry for β-HB detection in the presence of common interferences in ISF was assessed. Elevated β-HB levels occur in individuals with T1D who have high glucose levels. Therefore, the selectivity of the β-HB sensor was evaluated against varying glucose concentrations, as well as common electrochemically active analytes, including ascorbic acid (0.2 mM), uric acid (0.2 mM), and lactate (5 mM) (FIGS. 2L-2N). The normalized difference of the catechol peak current in interfering analytes compared to the 0 mM β-HB has an average of 0.81×10-3, while the average normalized difference of the quinone peak current is 50.1×10-3. Dunnett's one-way ANOVA analysis further verified the high selectivity. Since catechol oxidation provides a better sensitivity and selectivity compared to quinone reduction, catechol oxidation was chosen for detecting β-HB.

The Dopamine's quinone moieties were shown to serve as the redox mediator for detecting β-HB. However, at the physiological pH of ISF, DA, with a pKa of 8.9, is primarily in its protonated catechol form. The activity of tyrosinase might also be impeded while confined in the hydrogel network compared to its free form in solution, resulting in less available quinone moieties. To ensure enough supply of Quinones, an initial pre-oxidation step was performed via applying a positive potential to convert the catechol to quinone. The resulting quinone readily reacts with nearby NADH molecules. By calculating the ratio of catechol oxidation peak currents from the pre-oxidation and detection steps (I_{Detection}/I_{Pre-oxidation}), the amount of catechol recovered after reacting with NADH was observed. To establish the optimal wait time between the pre-oxidation and detection steps that yields the optimum response, the effect of 5-, 10-, and 15-second wait time in a 10 mM DA solution containing 0, 1, and 2.5 mM NADH was evaluated. FIG. 3A illustrates the pre-oxidation (dashed lines) and detection (solid lines) square wave voltammogram (SWV) curves with catechol oxidation at 0.3 V at different NADH concentrations. The catechol oxidation was observed to increase with increasing NADH concentration (FIG. 3B). A higher catechol recovery, measured by the I_{Detection}/I_{Pre-oxidation} ratio, was observed when the wait time was increased from 5 to 10 seconds (FIG. 3C). No substantial rise was observed when the wait time was extended from 10 to 15 seconds, suggesting that a 10-second wait time is adequate to detect changes in NADH concentration. In addition, the 5-second wait time exhibited a lack of clear linearity and notably higher standard deviation. Therefore, a 10-second wait time was implemented for the β-HB detection. Subsequently, the detection protocol is a 3-step process (FIG. 3D): (1) pre-oxidation step at 0.3V to convert catechol to quinone, (2) a 10-second wait time to allow quinone-NADH interaction, and (3) detection step at 0.3V to detect the amount of catechol produced. This method also helps account for sensor-to-sensor variability, ensuring accurate quantification of β-HB.

### Example 3. Microneedle Analyte Sensing Device Fabrication and Characterization

Upon successful validation of DA mediated-HB sensing, the sensing components were integrated into an HMN patch for wearable ketone sensing. HMN patches were fabricated through a micromolding process adapted from a previous work (FIG. 4A). Briefly, DA-HA was first dissolved in a solution of PEDOT:PSS (DHP solution). Then, a mixture of HBD, tyrosinase, and NAD+ was added to the DHP solution and mixed thoroughly. Upon adding tyrosinase, the polymeric mixture started to crosslink and was subsequently transferred to the molds. Finally, the HMN patches were kept at room temperature overnight to dry.

For successful skin penetration, the sharpness and mechanical strength of HMNs are critical. To investigate the impact of DA conjugation rate on needle integrity and structure, HMN patches were fabricated by combining HBD, tyrosinase, NAD+, and DHP solution with DA-HA hydrogel possessing 10% DA (HMN (10%)) or 17% DA (HMN (17%)). Scanning electron microscopy (SEM) images showed that both conjugation rates produced intact and sharp needles (FIG. 4B). However, dynamic mechanical analysis (DMA) that measures the patches' mechanical strength showed that the HMN (10%) patches are more flexible compared to the HMN (17%) patches, thus making the HMN (17%) more suitable for skin penetration (FIG. 4C). The effect of tyrosinase on the mechanical strength of patches fabricated with a 17% DHP solution (DHP+tyrosinase and DHP-tyrosinase) was studied (FIG. 4C). A threshold of 0.3-0.4 (N per needle) is required to have a successful needle insertion into the skin and all the samples showed sufficient mechanical strength for penetration. However, the needle breakage was observed only for the “DHP-tyrosinase” patch at higher forces, verifying the importance of tyrosinase to produce a mechanically strong patch. Considering the physical properties and mechanical strength of the patches, a dopamine conjugation rate of 17% was selected for the fabrication of the HMN-WE of microneedle analyte sensing device.

An important characteristic of microneedle analyte sensing device that enable ISF extraction is their porous structure and swelling capability. The swelling of HMN patches with different compositions (DHP−tyrosinase, DHP+tyrosinase, and HMN (17%)) was characterized by applying the patches on agarose skin mimicking hydrogel for 2 hours. DHP-tyrosinase patches showed ˜2.5 times higher swelling ratio compared to other two samples, confirming that tyrosinase should be present to form a highly crosslinked hydrogel (FIG. 4D). The swelling of the HMN (17%) (or HMN-WE) patches was studied after application on agarose hydrogel at different time points, revealing that the HMN patch reaches its maximum swelling after 30 minutes of insertion (FIG. 4E). SEM images showed a porosity of 60.8%, 39.8%, and 47.1% for DHP-tyrosinase, DHP+tyrosinase, and HMN-WE patches, respectively. Congruent with swelling results, DHP-tyrosinase showed higher porosity compared to DHP+tyrosinase and the HMN-WE patch, further verifying the tyrosinase's role in generating a highly crosslinked patch (FIG. 4F). Next, the electrical conductivity of the HMN-WE was examined, which is crucial for the patches to function effectively as the working electrode. The conductivity of the HMN-WE patches was measured in two different states: dried and swelled after application on agarose hydrogel for 2 hours. It was observed that the conductivity of HMN patches underwent a significant rise in the swelled state (1.32×106 μS cm−1) compared to the dried state (14.79 μS cm−1). As previously reported, some non-conductive PSS segments were washed out of the patch after swelling resulting in an increase in the PEDOT to PSS ratio (FIGS. 4G and 4H), thus enhancing the patch's conductivity.

### Example 4. Ketone Sensing Using an ex Vivo Skin Model

The microneedle analyte sensing device's capability as a wearable microneedle analyte sensing device on porcine skin equilibrated with various β-HB concentrations was evaluated (FIG. 5A). The HMN-WE patch was attached to a laser-induced graphene (LIG) for electrical connection and held together with MN-RE and MN-CE using Tegaderm. The HMN-WE successfully penetrated through the porcine skin and immediately started to swell and absorb the β-HB solution. The performance of the microneedle analyte sensing device in the presence of β-HB concentrations ranging from 0 to 1.5 mM was assessed. It is important to note that β-HB concentrations exceeding 0.6 mM signify ketosis and levels higher than 1.5 mM are an indicator of DKA. FIGS. 5B and 5C show the chronoamperometry curves corresponding to pre-oxidation (dashed lines) and detection (solid lines) steps.

The generated calibration curve (FIG. 5D) shows a linear correlation between the sensor response (I_{Detection}/I_{Pre-oxidation}) and β-HB concentration with a sensitivity of 0.08 mM−1. Most importantly, the microneedle analyte sensing device has the capability to notify individuals of the early onset of DKA when their ketone levels are slightly elevated, within the range of 0.6 to 1.5 mM. The microneedle analyte sensing device's performance at pH 7.0 and 7.3, the pertinent pH range corresponding to the mild and moderate severity levels of DKA was further evaluated. No significant differences were observed in the sensor response (I_{Detection}/I_{Pre-oxidation}) between pH 7.0 and 7.3, demonstrating the stable sensor performance in healthy and mild-moderate DKA patients.

An important characteristic of a wearable biosensor is its ability for continuous, long-term monitoring, relying on limited release of sensing components. Thus, ensuring efficient entrapment within the crosslinked hydrogel network is crucial. HBD and NAD+ leaching by fabricating HMN patches with and without tyrosinase on agarose hydrogel covered with a parafilm layer to mimic the skin's stratum corneum was examined (FIG. 5E). NAD+ release study using patches with tyrosinase showed a minimal release of 0.27% and 0.95% per patch after 1- and 10-hours application, respectively. However, almost all the NAD+ was released in the absence of tyrosinase, confirming tyrosinase's importance for NAD+ confinement in the hydrogel network. With a similar experimental set up, an enzyme release study revealed a minimal release of 0.07% and 0.19% per patch after 1 or 10 hours of application on agarose hydrogel, respectively, verifying successful enzyme entrapment. It is important to highlight that the agarose hydrogel contains higher water content compared to the skin, suggesting reduced swelling and less release in real-world skin applications. Next, the microneedle analyte sensing device's stability on porcine skin and conducted measurements every 10 minutes for 2 hours was examined (FIG. 5F). The sensor exhibited stable and reliable performance over time, as evidenced by a minimal sensor response change of less than 2%. The experiment was stopped at 2 hours because the porcine skin started to decompose when left in ambient conditions at room temperature.

A series of additional experiments was conducted to examine the mechanical stability and skin compatibility of the microneedle analyte sensing device. The microneedle analyte sensing device was applied and fixed with Tegaderm on porcine skin equilibrated in 1.5 mM β-HB overnight. After 100 consecutive cycles of twisting and bending (FIGS. 5G and 5H), electrochemical measurement was performed in three different states, including normal, bent, and twisted states. No significant differences in the sensor response (I_{Detection}/I_{Pre-oxidation}) were observed in all three states (FIG. 5I) demonstrating the stable performance of the microneedle analyte sensing device under different mechanical tensions. FIGS. 5J and 5K show the chronoamperometry curves corresponding to the pre-oxidation (dashed lines) and detection (solid lines) at different states. Additionally, to further validate the microneedle analyte sensing device's electrochemical stability after being subjected to bending and twisting, electrochemical measurements were performed after successive bending and twisting cycle for 10 cycles. As shown in FIG. 5I, the sensor response only showed a 5% deviation after each cycle, which further confirms the mechanical stability of the microneedle analyte sensing device. To study the tissue adhesive properties of the microneedle analyte sensing device, a lap shear test was performed. Fresh porcine skin was used as a skin model due to its higher resemblance to human skin, and the standard stress-distance curve was obtained. The HMN's maximum adhesion strength was 0.4 MPa, which is higher than reported values in other works. The sudden drop to 0.4 MPa represents the detachment of microneedle analyte sensing device from porcine skin.

### Example 5. In Vivo Validation of Microneedle Analyte Sensing Device

Upon establishing the capability of microneedle analyte sensing device for measuring β-HB in the skin model, the sensor capability for in vivo monitoring using a rat model of T1D was examined. Prior to the animal experiment, the biocompatibility and cytotoxicity of the HMN-WE via MTT assay after exposing the fibroblast cells to the materials used in the fabrication of HMN-WE was tested. The cell viability was not significantly affected (cell viability=93.8%), demonstrating the biocompatibility of HMN-WE patch. The biocompatibility of MN-CE and MN-RE was studied in the previous works. To assess the skin penetration capability of HMN-WE, the HMN patches were applied on the rat's back for 2 minutes. Right after the patch removal, the HMN traces were clear demonstrating the successful skin penetration. Rat skin samples were collected after 5 minutes of application for further analysis with hematoxylin and eosin (H&E) staining. As seen in FIG. 6A, a clear needle cavity of 90.5 μm was observed indicating successful penetration of HMNs through the skin's dermis layer. However, due to skin healing upon patch removal, the measured needle cavity depth is less than the HMN height.

Fasted diabetic rats with a ketone level of more than 2.5 mM were chosen for the in vivo experiments. An increase in blood ketone levels was observed after the rats were sedated under isoflurane gas. This may be attributed to alterations in the rats' metabolic processes under isoflurane gas. The HMN-WE along with the MN-RE and the MN-CE, were applied on the shaved back of rats and fixed using Tegaderm tape (FIG. 6B). The electrochemical signal measurements were then acquired after 60 minutes, allowing the HMN-WE to reach its maximum swelling. Following the baseline measurement, the rats received a dose of 10U insulin to reduce their blood ketone levels, and electrochemical measurements were subsequently obtained every 10-15 minutes for 60-80 minutes. Reference blood ketone samples were also obtained from the rat tail and measured using a ketone meter before each electrochemical measurement. As shown in FIGS. 6C-6G, a decrease in blood ketone levels resulted in a decrease in sensor response over time. Comparatively, HMN patches with no HBD enzyme did not show any response over time. To evaluate the microneedle analyte sensing device's performance for monitoring increasing ketone levels, the microneedle analyte sensing device was tested using the T1D rats upon sedation with isoflurane gas without insulin injection where an increase in ketone level was observed. The microneedle analyte sensing device enables continuous measurement of both increasing and decreasing ketone levels.

### Example 6

In some embodiment, the disclosed microneedle analyte sensing device is an electrochemical biosensor with three-electrode system, comprised of a pseudo reference electrode (Ag/AgCl paste), a gold counter electrode, and a gold working electrode. The working electrode surface was modified with MWCNTs paste in order to increase the surface area as well as facilitating the redox mediator polymerization. Then the working electrode surface was modified with electro polymerization of Toluidine Blue-O as a redox mediator. The TBO polymerization involves different steps, namely polymerization, washing, and stabilization, and each of these steps serves a purpose to have a stable platform. The TBO's function as a redox mediator in NADH sensing was verified by narrowing the overpotential in the NADH oxidation peak. It is observed that in the presence of TBO, NADH oxidation potential shifted from 1 V to 0.15 V (vs. Ag/AgCl), further verifying the enhancement in sensing of NADH without dependency to higher potentials (FIG. 7E). Then, the chronoamperometry technique (0.15 V, t=40 s) was used for the future studies. The responses of TBO modified electrode to various concentrations of NADH were evaluated, for which a linear behaviour was observed with high reproducibility, and the sensitivity of 1.3086 (uA/mM) was obtained. Next, the specificity of the NADH sensor was challenged with different interferents and a strong specificity was observed to NADH in the presence of such interferents as ascorbic acid, uric acid, lactate, glucose, sucrose, NaCl, and MgCl2 abundantly found in the body by minimizing the interferent signals (FIG. 7F). In addition, the stability of TBO mediator was studied. To do so, TBO modified electrodes were evaluated against 1 mM NADH every 15 minutes, and stable responses were obtained with a maximum 15% change over 2 hrs (FIG. 7I).

### Example 7

Having verified the performance of NADH sensor, β-HBD along with NAD⁺ were immobilized on TBO modified electrode. β-HBD enzyme oxidizes β-HB to AcAc with concurrent reduction of NAD⁺ cofactor to NADH which will be oxidized by TBO, through which NADH will be detected and NAD⁺ will be recovered and reused. To measure the β-HB level, chronoamperometry technique was utilized at 0.15 V. Next, the disclosed microneedle analyte sensing device's performance was recorded for different concentrations of β-HB, and a linear response was observed in the range of interest with a sensitivity of 188.27 (nA/mM) (FIG. 8C). Additionally, a specificity of disclosed microneedle analyte sensing device was evaluated, for which the disclosed microneedle analyte sensing device performance was challenged with potential interferents. It was observed that the β-HB biosensor can accurately differentiate the signal originated from β-HB in the presence of different interferents present in the body, including 5 mM glucose, 5 mM sucrose, 5 mM lactate, 0.2 mM ascorbic acid, 0.2 mM uric acid, 150 mM NaCl, and 150 mM MgCl₂. The biosensor's shelf life is another important factor that should be taken into account in designing and fabrication of the biosensor. Here, a packaging method by a bilayer Aluminium foil laminated with a polymer was used to avoid not only the oxygen inhibition but also any adverse impact caused by light exposure to deteriorate TBO. The packaging method could preserve the biosensor activity up to two weeks (FIG. 8E) without any significant decrease in its performance.

### Example 8

The disclosed microneedle analyte sensing device implementation. A disclosed microneedle analyte sensing device patches consist of 100 needles, each with a 200 μm base and an 850 μm height. The disclosed microneedle analyte sensing device patches were fabricated using highly crosslinked (HC) Methacrylate Hyaluronic Acid (MeHA) hydrogel, which was crosslinked in the presence of MBA and a photo-initiator (PI) under UV light exposure. The characterization of MeHA microneedles involved various techniques. SEM imaging revealed sharp and uniform needles. Furthermore, the impact of crosslinking duration on swelling and mechanical properties was evaluated. Swelling data indicated that 10 minutes of crosslinking produced the same swelling properties as 15 minutes. The required force for skin penetration is 0.3 N per needle, and all microneedles could withstand this force threshold; however, the compression data showed that the microneedle with 5 minutes of crosslinking had lower strength compared to those with 10 and 15 minutes of crosslinking. Nevertheless, 15 minutes was selected as the optimal crosslinking time due to its demonstration of the highest Young's modulus (FIG. 9D). Subsequently, microneedle analyte sensing device patches were attached to the ketone biosensor electrode using low-crosslinked (LC) MeHA, and the microneedle biosensors were employed in various studies, including in vitro calibration and in vivo experiments (FIG. 9E).

### Example 9

After validating the ketone biosensor performance in in-vitro studies with and without microneedle, in vivo studies were performed to evaluate the disclosed microneedle analyte sensing device performance on the live animal model. To this end, healthy rats were used first. The mixture of isoflurane/oxygen was used for both induction and maintenance of rats under anesthesia. The disclosed microneedle analyte sensing device were applied on the dorsal skin of rats after hair removal. Then, a 25-gauge catheter was inserted into the rat's tail and fixed with medical tape, then was connected to the syringe pump. The ketone solution with 1.5 M (β-HB in saline) concentration was infused intravenously through the rat's tail, and subsequently distributed in the blood circulating system. Blood samples were taken from the rat's tail, and its ketone level was measured with the Freestyle Libre ketone meter, and concurrently chronoamperometry measurements were performed every 5 minutes to cross validate the signals with blood ketone level (FIGS. 10D, 10E, and 10F). In all rat experiments, our electrodes were able to show the rises along with the falls in ketone level. It was observed that there is 5-10 minutes of delay in ISF ketone compared to the blood ketone, which is in agreement with other studies as well. This delay can be attributed to the mass transfer between blood and ISF and the mass transfer in the hydrogel microneedle. There was a patch-to-patch variation originating from fabrication steps; to overcome this issue, the response currents were normalized to the initial current response, and more consistency was observed between the different experiments. Additionally, histology studies (H&E staining) were performed, and it was observed that hydrogel microneedle was able to break the stratum corneum layer and penetrate the skin to reach interstitial fluid.

### Example 10

Having verified the microneedle analyte sensing device on the rat animal model for continuous monitoring, the pig animal model, which is a large animal model and more resembles humans in terms of physiology was used to evaluate the disclosed microneedle analyte sensing device. To this end, electrodes were applied on the pig's ear after hair removal, and 50 minutes of swelling time was required so that the microneedles got saturated and the ISF reached the electrode surface. In the first series of experiments, 3 different pigs were used (FIGS. 11D, 11E, and 11F). First, the pigs were sedated and later, they were anesthetized using isoflurane. For the first series of studies, the stability of electrode responses was assessed, and stable responses were observed for the electrodes. The blood ketone levels during this series of studies were zero (measured with Freestyle Libre Ketone Meter). The baseline current level differs for various pigs, showing that individuals can exhibit different physiological responses in the given conditions, underscoring the importance of personalized medicine. However, in the second series of studies, the microneedle analyte sensing device was challenged with continuous change in blood ketone. First, oral gavage of ketone supplements (500 mg/kg BM, deltaG Oxford) through intubation was tried to increase the ketone level in pigs; however, it was unsuccessful. This can be attributed to the anesthesia state, which may slow down the speed of the digestion process, or the initial insulin release that consumed the administrated ketone. Later, ketone (β-HB) infusion to healthy pig blood, to increase and decrease the ketone level was tested. To this end, a catheter was inserted into the ear of the pig and connected to the syringe pump through which the ketone (1.5 M dissolved in sterile saline serum) was infused into the pig blood. The blood sampling was done every 5 minutes from the leg, and its ketone level was measured using the Freestyle Libro ketone meter with the simultaneous electrochemical measurement using chronoamperometry technique every 5 minutes. This experiment was performed 4 times with at least one week time intervals; an increasing and a decreasing pattern was observed in the blood ketone. The microneedle analyte sensing device was able to measure ISF ketone with a very similar dynamic to blood ketone, although a delay of 10-20 minutes was observed in all the experiments. Baseline correction was done for the electrodes whenever required to minimize any discrepancies caused by patch-to-patch variation. As shown in FIG. 11J, the ketone injection was performed twice at different time points, and microneedle analyte sensing device could successfully monitor the increasing and decreasing patterns even at multiple steps, further verifying the microneedle analyte sensing device performance. Also, the skin recovery process was evaluated for the pig, and it was observed that the needle traces vanished in 20 minutes, underlining the fast recovery and minimum adverse impact on the skin. Next, a skin tissue sample was collected for histology purposes upon euthanization. The H&E staining was performed, and the breakage in the stratum corneum was observed, which verifies the ability of the hydrogel microneedle in rupturing the stratum corneum of pig skin and reaching the ISF. It is worth mentioning that pig's skin is closely similar to humans', indicating that the microneedle analyte sensing device with current design can potentially be applicable in human studies.

### Example 11

A preliminary in-human testing of the microneedle analyte sensing device is shown in FIGS. 11A and 11B. The microneedle analyte sensing device was applied to the forearm of a healthy volunteer and used for continuous monitoring of ketones after the consumption of a ketone solution. The microneedle analyte sensing device provided continuous measurements, while blood samples were collected every 10 minutes for blood-based measurement using a ketone meter for cross-validation.

Using the collected measurements, a data-driven decision-tree-based machine learning (ML) framework using the stochastic gradient boosting algorithm was developed to predict the ketone levels. The ML model was also able to improve the noisy signal often obtained from in vivo experiments and circumvent the need for a pre-calibration step before usage. Specifically, the

ML model revealed a time-lag between the ISF ketone measured by the microneedle analyte sensing device and the blood ketone. The time-lag is mainly caused by variations in transport efficiencies between the ISF and the blood in circulation. For individual rats, varying delays ranging from 9-28 minutes were found. FIG. 6H shows that the ketone concentration measured by the sensor is well-correlated with the reference blood ketone considering the time-lags. The differences between the time-lags in individual rats clearly show the variability in ketone kinetics, even under controlled conditions with genetically similar animals. This has important implications for human patients, who are genetically diverse and have different environmental conditions, intensifying this inter-individual variability. Thus, these results highlight the necessity of personalized ketone monitoring. Previous studies reported the use of 1.5 mM as a cutoff ketone level to compare the accuracy performance of CKM sensors to blood ketone since it signifies a high-risk ketoacidosis. A mean absolute difference (MAD) of 0.184 mM for ketone levels <1.5 mM and an mean absolute relative difference (MARD) of 7.68% for ketone levels of ≥1.5 mM, was calculated, comparable to another CKM sensor developed by Abbott Inc. (Table 1).

### Discussion and Significance

Diabetic ketoacidosis, a severe complication of T1D, necessitates the development of microneedle analyte sensing device. Here, a skin-compatible microneedle analyte sensing device for continuous ketone monitoring was reported and validated. The dopamine molecules that are covalently linked to the polymer structure of the HMN patch serve multiple functions necessary to develop a robust β-HB sensor. First, the catechol-quinone chemistry inherent in dopamine acts as a redox mediator for NADH measurement, a byproduct of β-HB catalysis, at a low potential to minimize fouling and interfering signals. Furthermore, in conjunction with tyrosinase, dopamine facilitates the formation of a tightly crosslinked HMN patch with enhanced mechanical strength. Dynamic mechanical analysis demonstrated that the microneedle analyte sensing device exhibited a high Young's modulus to ensure successful skin penetration. The highly crosslinked HMN also minimized the leaching of the sensing components within the polymeric network, which enhanced the stability of the sensor during prolonged measurements. This is particularly vital for dehydrogenase-based enzymatic sensors that require additional redox mediators and co-factors. Furthermore, the HMN's flexibility due to ISF absorption upon skin application is mechanically compatible with the skin and the biocompatible DAHA-based material also circumvents the potential needle breakage issue that solid MNs face.

Table 1. Accuracy analysis of the predicted ISF ketone level against the reference blood ketone. Mean absolute difference (MAD) and mean absolute relative difference (MARD) calculated for ketone levels <1.5 mM and >=1.5 mM, respectively.

TABLE 1

| | | | Percentage | Percentage |
|---|---|---|---|---|
| | | | within 0.225 | within 0.3 |
| | Criteria | MAD/MARD | mM/20% | mM/30% |
| Normal or ketosis | <1.5 mM | 0.184 mM | 71.43% | 71.43% |
| Hyperketonemia | ≥1.5 mM | 7.68% | 88.89% | 96.30% |
| or DKA | | | | |

Ex vivo characterization of the microneedle analyte sensing device using a skin model showed that the sensor can accurately measure the ketone levels with a sensitivity of 0.08 mM−1 mM. Specifically, the sensor measures the ketone levels within the range of 0.6 mM−1.5 mM to monitor the early onset of DKA. Subsequent experiments in diabetic rats along with a ML model confirmed the microneedle analyte sensing device capacity to continuously monitor in vivo changes in ketone levels in real-time and highlighted its capability to accurately decipher the delay between ISF and blood measurements in individual rats, a crucial aspect for clinical adoption. Importantly, the microneedle analyte sensing device measurements closely matched those obtained with standard ketone meter considering the time-lag. The microneedle analyte sensing device was compared with recently published β-HB sensors. This comparison demonstrates that the microneedle analyte sensing device is capable of continuously monitoring β-HB using the catechol-quinone-mediated chemistry.

Overall, the microneedle analyte sensing device successfully demonstrated the first continuous monitoring of ISF ketone in diabetic rats. The next steps towards the clinical implementation of microneedle analyte sensing device involve further validation of the system.

The first aspect is to evaluate the extended working capabilities of the sensor beyond a few hours to days. Prior to testing on healthy and T1D patients, it would be beneficial to assess the sensor's performance and its safety using a pig model, whose skin physiology closely resembles that of human skin. Lastly, the microneedle analyte sensing device must be integrated with an HMN-CGM sensor to develop a CGM-CKM device for comprehensive diabetes monitoring.

Beyond diabetes management, the microneedle analyte sensing device has the potential to be utilized for the management of ketogenic diets and can also be used by athletes to monitor their ketone levels during intense exercise. The versatility of the catechol-quinone sensing chemistry can potentially serve as the foundation of other sensors, such as lactate and alcohol. Therefore, multiplexed monitoring systems that employ the catechol-quinone chemistry can be developed to track other health conditions, demonstrating its broad application for continuous monitoring technologies.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

Thus, those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

### REFERENCES

The following references are incorporated herein by reference in their entirety for all purposes.
1. H. Teymourian, C. Moonla, F. Tehrani, E. Vargas, R. Aghavali, A. Barfidokht, T. Tangkuaram, P. P. Mercier, E. Dassau, J. Wang, Anal. Chem. 2020, 92(2), 2291-2300.
2. C. Moonla, M. Reynoso, A. Casanova, A. Chang, O. Djassemi, A. Balaje, A. Abbas, Z. Li, K. Mahato, J. Wang, ACS Sens. 2024.
3. N. J. Forrow, G. S. Sanghera, S. J. Walters, J. L. Watkin, Biosens. Bioelectron. 2005, 20(8), 1617-1625.
4. H. Teymourian, C. Moonla, F. Tehrani, E. Vargas, R. Aghavali, A. Barfidokht, T. Tangkuaram, P. P. Mercier, E. Dassau, J. Wang, Anal. Chem. 2020, 92(2), 2291-2300.
5. B. Ge, Y. Tan, Q. Xie, M. Ma, S. Yao, Sens. Actuators, B. 2009, 137(2), 547-554.
6. S. Odinotski, K. Dhingra, A. GhavamiNejad, H. Zheng, P. GhavamiNejad, H. Gaouda, D. Mohammadrezaei, M. Poudineh, Small. 2022, 18(45), 2200201.
7. P. GhavamiNejad, A. GhavamiNejad, H. Zheng, K. Dhingra, M. Samarikhalaj, M. Poudineh, Adv. Healthcare Mater. 2023, 12(1), 2202362.
8. Y. Liu, J. Liu, S. Chen, T. Lei, Y. Kim, S. Niu, H. Wang, X. Wang, A. M. Foudeh, J. B.-. Tok, Z. Bao, Nat. Biomed. Eng. 2019, 3(1), 58-68.
9. J. Kang, K. Y. Kim, S. Kim, H. Hong, B. Bae, S. Kang, W. Lee, Device. 2023, 1(4).
10. W. Lee, S. Jeong, Y. Lim, H. Lee, J. Kang, H. Lee, I. Lee, H. Han, S. Kobayashi, M. Tanaka, B. Bae, Sci. Adv. 2021, 7(48), eabi6290.
11. J. Jiang, R. Wang, M. Wang, K. Gao, D. D. Nguyen, G. Wei, J. Chem. Inf. Model. 2020, 60(3), 1235-1244.

What is claimed is:

1. A microneedle analyte sensing device for continuously monitoring and measuring an analyte in a biological fluid of a user, the microneedle analyte sensing device comprising:
a plurality of microneedles operable to penetrate a surface of a biological tissue of the user and contact the plurality of microneedles with the biological fluid when the microneedle analyte sensing device is attached to the surface of the biological tissue,
wherein:
at least one microneedle of the plurality of microneedles is a working electrode that detects an electrical signal generated from an electrochemically mediated enzymatic reaction with the analyte in the biological fluid of the user;
at least one microneedle of the plurality of microneedles is a counter electrode; and
at least one microneedle of the plurality of microneedles is a reference electrode.

2. The microneedle analyte sensing device of claim 1, wherein at least one of:
the at least one microneedle that is the working electrode is made of a hydrogel;
the at least one microneedle that is the counter electrode is made of ultraviolet (UV)-cured epoxy and coated with metal nanoparticles, wherein the metal nanoparticles comprise platinum, silver, gold, palladium, or combinations thereof;
the at least one microneedle that is the reference electrode is made of ultraviolet (UV)-cured epoxy and coated with silver-silver chloride (Ag/AgCl); or a
combination thereof.

3. The microneedle analyte sensing device of claim 2, wherein at least one of:
the hydrogel comprises at least one of hyaluronic acid, methacrylated hyaluronic acid, gelatin, methacrylated gelatin, alginate, methacrylated alginate, chitosan, methacrylated chitosan, collagen, methacrylated collagen, or a combination thereof;
the at least one microneedle that is the working electrode comprises enzymes integrated in the hydrogel, and wherein the enzymes comprise at least one of beta-hydroxybutyrate dehydrogenase (HBD), tyrosinase, or a combination thereof;
the at least one microneedle that is the working electrode comprises a HBD cofactor integrated in the hydrogel, wherein the HBD cofactor comprises nicotinamide adenine dinucleotide (NAD+);
the at least one microneedle that is the working electrode comprises a redox mediator integrated in the hydrogel to facilitate electron transfer in the electrochemically mediated enzymatic reaction;
the at least one microneedle that is the working electrode comprises an electrically conductive material integrated in the hydrogel to increase electrical conductivity of the at least one microneedle; or
a combination thereof.

4. The microneedle analyte sensing device of claim 2, wherein the microneedle analyte sensing device detects the electrical signal generated from the electrochemically mediated enzymatic reaction in situ.

5. The microneedle analyte sensing device of claim 1, wherein the analyte is ketone, beta-hydroxybutyrate, lactate, acetone, or glucose.

6. The microneedle analyte sensing device of claim 1, wherein the biological fluid is interstitial fluid, transdermal fluid, or blood.

7. The microneedle analyte sensing device of claim 1, wherein at least one of:
the plurality of microneedles are disposed on a substrate or within a substrate that the plurality of microneedles are operable to penetrate;
the microneedle analyte sensing device is integrated into a transdermal patch;
the biological tissue is skin or stratum corneum; or
a combination thereof.

8. The microneedle analyte sensing device of claim 3, at least one of:
the redox mediator comprises at least one of dopamine, conjugated dopamine, functionalized dopamine, crosslinked dopamine, metal-complexed dopamine, poly toluidine blue O (PTBO), toluidine blue O (TBO), or combinations thereof;
the electrically conductive material comprises at least one of poly(3,4-ethylenedioxythiophene): polystyrene sulfonate (PEDOT:PSS), a metal nanoparticle, graphene, MXene, conductive polymer, polyaniline, polypyrrole, ionomer, carbon nano tube, or a combination thereof;
the electrochemically mediated enzymatic reaction is detectable using amperometry, impedimetry, conductometry, voltammetry, or potentiometry; or
a combination thereof.

9. The microneedle analyte sensing device of claim 1, wherein the microneedle analyte sensing device is a continuous ketone monitoring (CKM) sensor.

10. An analyte sensing device comprising:
the microneedle analyte sensing device of claim 1; and
an electrical circuit electrically comprising a data processing unit,
wherein:
the electrical circuit is connected to the microneedle analyte sensing device and processes (e.g., programmed to process) the electrical signal generated by the electrochemically mediated enzymatic reaction; and
the data processing unit comprises a processor and a memory, and processes (e.g., configured to process) the electrical signal as data representative of one or more parameters of the analyte.

11. The analyte sensing device of claim 10, further comprising a wireless communication unit in communication with the electrical circuit to transmit a processed signal to a user interface, wherein the user interface comprises a smartphone, a personal computer, a laptop, a tablet, a wearable device, a smart home device, an Internet of Things (IoT) device, or a combination thereof.

12. The analyte sensing device of claim 10, wherein the analyte sensing device is a continuous ketone monitoring (CKM) sensor.

13. A method for measuring an analyte within a biological fluid of a user, the method comprising:
providing a microneedle analyte sensing device, wherein the microneedle analyte sensing device comprising a plurality of microneedles operable to penetrate a surface of a biological tissue of the user and contact the plurality of microneedles with the biological fluid when the microneedle analyte sensing device is attached to the surface of the biological tissue;
placing the microneedle analyte sensing device on the surface of the biological tissue of the user to contact (e.g., transdermally contact) the plurality of microneedles with the biological fluid;
applying an electrical stimulus signal to at least one microneedle of the plurality of microneedles;
detecting an electrical signal arising by an electrochemically mediated enzymatic reaction with the analyte in the biological fluid of the user exposed to the at least one microneedle; and
determining a concentration of the analyte based on the electrical signal.

14. The method of claim 13, wherein:
at least one microneedle of the plurality of microneedles is a working electrode that detects the electrical signal generated from the electrochemically mediated enzymatic reaction with the analyte in the biological fluid of the user;
at least one microneedle of the plurality of microneedles is a counter electrode; and
at least one microneedle of the plurality of microneedles is a reference electrode.

15. The method of claim 13, at least one of:
the plurality of microneedles are disposed on a substrate or within a substrate that the plurality of microneedles are operable to penetrate;
the microneedle analyte sensing device is integrated into a transdermal patch;
the biological tissue is skin or stratum corneum;
the electrical signal is transferred through the at least one microneedle to an electrical circuit;
the method further comprises sending the electrical signal from the electrical circuit to a data processing unit, wherein the data processing unit comprises a processor and a memory, and processes (e.g., configured to process) the electrical signal as data representative of one or more parameters of the analytes;
the method further comprises sending the electrical signal from the data processing unit to a wireless communication unit in communication with the electrical circuit to transmit a processed signal to a user interface, wherein the user interface comprises a smartphone, a personal computer, a laptop, a tablet, a wearable device, a smart home device, an Internet of Things (IoT) device, or a combination thereof; or
a combination thereof.

16. A method of manufacturing a microneedle analyte sensing device, the method comprising:
preparing a composition for a working-electrode by mixing a hydrogel, at least one enzyme, at least one enzyme cofactor, a redox mediator, and an electrically conductive material; and
applying or injecting a composition for a working-electrode to a micro-mold for microneedles of a working electrode, wherein the composition for the working-electrode comprises a hydrogel, at least one enzyme, at least one enzyme cofactor, a redox mediator, and an electrically conductive material.

17. The method of claim 16, wherein at least one of:
the method further comprises preparing the composition for the working-electrode by mixing the hydrogel, the at least one enzyme, the at least one enzyme cofactor, the redox mediator, and the electrically conductive material.
the method further comprises preparing a counter electrode, comprising:
applying or injecting ultraviolet (UV)-curable epoxy to a micro-mold for microneedles of the counter electrode;
curing the UV-curable epoxy of the counter electrode, optionally via ultraviolet light;
coating the counter electrode with metal nanoparticles, wherein the metal nanoparticles comprise platinum, silver, gold, palladium, or combinations thereof;
the method further comprises preparing a reference electrode, comprising:
applying or injecting ultraviolet (UV)-curable epoxy to a micro-mold for microneedles of the reference electrode;
curing the UV-curable epoxy of the reference electrode, optionally via ultraviolet; and
coating the reference electrode with silver-silver chloride (Ag/AgCl);
the hydrogel comprises at least one of hyaluronic acid, methacrylated hyaluronic acid, gelatin, methacrylated gelatin, alginate, methacrylated alginate, chitosan, methacrylated chitosan, collagen, methacrylated collagen, or combinations thereof;
the at least one enzyme comprise at least one of beta-hydroxybutyrate dehydrogenase (HBD), tyrosinase, or a combination thereof;
the at least one enzyme cofactor comprises nicotinamide adenine dinucleotide (NAD+);
the redox mediator comprises dopamine, conjugated dopamine, functionalized dopamine, crosslinked dopamine, metal-complexed dopamine, poly toluidin blue O (PTBO), toluidine blue O (TBO), or combinations thereof;
the electrically conductive material comprises at least one of poly(3,4-ethylenedioxythiophene): polystyrene sulfonate (PEDOT:PSS), a metal nanoparticle, graphene, MXene, conductive polymer, polyaniline, polypyrrole, ionomer, carbon nano tube, or a combination thereof; or
a combination thereof.

18. The method of claim 16, wherein at least one of:
the microneedles of the working electrode are homogeneous throughout the microneedles;
the microneedles of the counter electrode are homogeneous throughout the microneedles;
the microneedles of the reference electrode are homogeneous throughout the microneedles;
the method further comprises forming one or more molds corresponding to shapes of (i) the microneedles of the working electrode, (ii) the microneedles of the counter electrode, (iii) the microneedles of the reference electrode, or (iv) a combination thereof; or
a combination thereof.

19. A method of manufacturing a microneedle analyte sensing device, the method comprising:
applying a first composition to an electrode, wherein the first composition comprises a redox mediator, chitosan, and an electrically conductive material;
curing the first composition to generate a first layer;
applying a second composition to the first layer to generate a second layer on top of the first layer, wherein the second composition comprises at least one enzyme and at least one enzyme cofactor; and
applying a third composition to the second layer to generate a third layer on top of the second layer, wherein the third composition comprises chitosan.

20. The manufacturing method of claim 19, wherein at least one of:
the method further comprises preparing the first composition by mixing the redox mediator, the chitosan, and the electrically conductive material;
the method further comprises preparing the second composition by mixing the at least one enzyme and the at least one enzyme cofactor;
the electrode is a screen printed gold electrode;
the electrically conductive material comprises or is carbon nanotubes (e.g., multi-walled carbon nanotubes, single-walled carbon nanotubes, double-walled carbon nanotubes, or a combination thereof);
curing the first composition via cyclic voltammetry to generate a first layer;
the at least one enzyme comprise at least one of beta-hydroxybutyrate dehydrogenase (HBD), tyrosinase, or a combination thereof;
the at least one enzyme cofactor comprises nicotinamide adenine dinucleotide (NAD+);
the redox mediator comprises at least one of dopamine, conjugated dopamine, functionalized dopamine, crosslinked dopamine, metal-complexed dopamine, poly toluidin blue O (PTBO), toluidine blue O (TBO), or combinations thereof; or
a combination thereof.
